(19) United States
(12) Patent Application Publication
(10) Pub. No.: US 2025/0361368 A1
(43) Pub. Date: Nov. 27, 2025

(54) FOCUSED ULTRASOUND (FUS) CROSSLINKING AND PORE-GENERATION IN GRANULAR HYDROGELS

(71) Applicant: UNIVERSITY OF VIRGINIA PATENT FOUNDATION, Charlottesville, VA (US)

(72) Inventors: Natasha Claxton, Charlottesville, VA (US); Matthew Ryan DeWitt, Charlottesville, VA (US); Richard J Price, Charlottesville, VA (US); Christopher Bruce Highley, Crozet, VA (US)

(21) Appl. No.: 19/219,602

(22) Filed: May 27, 2025

Related U.S. Application Data
(60) Provisional application No. 63/652,159, filed on May 27, 2024.

(51) Int. Cl. C08J 3/24; A61L 27/18; A61L 27/22; A61L 27/52; A61L 27/54; C08J 3/28

(52) U.S. Cl. CPC C08J 3/242; A61L 27/18; A61L 27/222; A61L 27/52; A61L 27/54; C08J 3/28; A61L 2430/00; C08J 2371/08

(57) ABSTRACT

Provided herein is a method of preparing a crosslinked granular hydrogel, the method includes mixing a hydrogel microparticle having a first crosslinking group, a polymeric fiber having a second crosslinking group, an initiator, and a crosslinker to form a precursor composition. The method also includes applying focused ultrasound (FUS) to the precursor composition, whereby each of the first crosslinking group and the second crosslinking group reacts with the crosslinker, thereby the hydrogel microparticle and the polymeric fiber are crosslinked to form the granular hydrogel.

### CROSS-REFERENCE TO RELATED APPLICATIONS

The present application claims priority to U.S. Provisional Patent Application No. 63/652,159 filed on May 27, 2024, of which is hereby incorporated by reference in its entirety.

### STATEMENT REGARDING FEDERALLY SPONSORED RESEARCH

This invention was made with government support under R35GM147410 awarded by the National Institutes of Health and under 5T32GM136615 awarded by the National Institute of General Medical Sciences. The government has certain rights in the invention.

### FIELD OF THE INVENTION

The disclosed technology is generally directed to focused ultrasound responsive granular hydrogels and their use in enhancing particle-based hydrogel scaffolds for regenerative medicine and tissue engineering.

### BACKGROUND OF THE INVENTION

Particle-based hydrogels are a rapidly emerging class of materials that have applications in regenerative medicine, tissue engineering, and biofabrication (i.e., 3D (bio) printing). A macroscale (millimeter scale and larger) volume of these materials consists of individual microscale particles (generally 200 μm or less) in characteristic lengths. Typically, these particles are spherical, and the characteristic length is the diameter. Macroscale materials whose volumes consist of these microscale particles are sometimes referred to as granular materials, where the individual, discrete particles might be considered grains. In the case of particle-based hydrogels, the individual particles are generally microscale hydrogels (microgels) formed via emulsification processes (including batch emulsification or microfluidic particle generation). By concentrating these particles (removing much or all of the liquid between them), they come into physical contact. Macroscale, collective bulks can be stabilized by interparticle crosslinks or, when enough fluid between the particles is removed, by simple physical interactions between particles in jammed systems. In the latter case, bulk granular hydrogels are static when the stress applied to them is below a characteristic yield stress, but they will flow with case when stress applied to the bulk (or to a specific location in the bulk) exceeds the yield stress. These properties are extremely desirable in bioprinting (where materials must flow during extrusion and rapidly stabilize after) and in regenerative medicine applications where injectable materials are desirable. Additionally, porosity can be controlled through the amount of fluid included between particles, which has been seen to be advantageous in regenerating tissue. High porosity is important in applications of granular hydrogels where cells are incorporated withing the granular material or when the granular material is intended to support or promote cellular migration or ingrowth into its volume.

The mechanical integrity of a granular hydrogel is derived in large part from particle-particle interactions, often through chemical bonds between microparticles. When these bonds are weak, or when there is limited particle-particle contact, as in the case of increasingly porous granular materials, the structure of the granular material can be compromised, which can result in its falling apart or condensing and losing porosity. Thus, there remains a need for methods of making hydrogels that maintain stability and desirable mechanical properties at relatively high porosity. Such advanced hydrogel compositions would enable broader applications in therapeutic delivery, tissue engineering, and regenerative medicine interventions.

### SUMMARY OF THE INVENTION

In one aspect, the present disclosure provides a method of preparing a crosslinked granular hydrogel. The method includes mixing a hydrogel microparticle having a first crosslinking group, a polymeric fiber having a second crosslinking group, an initiator, and a crosslinker to form a precursor composition. The method further includes applying focused ultrasound (FUS) to the precursor composition, whereby each of the first crosslinking group and the second crosslinking group reacts with the crosslinker, thereby the hydrogel microparticle and the polymeric fiber are crosslinked to form the granular hydrogel. In some embodiments, the precursor composition further includes a removable particle, which upon removal changes the porosity of the granular hydrogel, and/or a viscosity promotor, which increases viscosity upon FUS-induced heating. In some embodiments, the method includes removing the removable particle, thereby changing the porosity of the granular hydrogel.

In another aspect, the present disclosure provides a method of regenerating a tissue in a subject. The method includes preparing a crosslinked granular hydrogel from a precursor composition injected into the subject, according to the preparation process described herein. The precursor composition has an embedded cell. The method further includes allowing the cell to grow in the subject, thereby regenerating a tissue from the cell.

In another aspect, the disclosure provides a method of delivering a drug to a subject. The method includes preparing a crosslinked granular hydrogel from a precursor composition injected into the subject, according to the preparation process described herein. The precursor composition has the drug embedded therein. The method further includes releasing the drug from the crosslinked granular hydrogel, thereby delivering the drug to the subject.

Another aspect of the present disclosure provides a crosslinked granular hydrogel produced by the preparation methods described herein. The disclosure further provides a scaffold including the crosslinked granular hydrogel.

In an aspect, the methods described herein provide a method of culturing cells. The method includes mixing the cells with the scaffold, thereby the cells are embedded in the scaffold, and culturing the cells embedded in the scaffold. The present disclosure further provides an implant including a crosslinked granular hydrogel produced from an injected precursor composition.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A-1C shows a schematic illustration of presumptive focused ultrasound crosslinking process for granular, fiber-containing hydrogel scaffolds. A. Polyethylene glycol (PEG) microgels containing excess acrylates, PEG fibers containing excess norbornenes, and gelatin microgels (I) are mixed with a tetra PEG-thiol solution, an initiator (potassium persulfate), and a thermo-responsive material (PDEGMA) (II). B. Upon focused ultrasound application, gelatin microgels begin to melt (I), generated radicals initiating crosslinking between the thiols and acrylates/norbornenes, and PDEGMA undergoes globule structural transformation upon heating to locally increase viscosity and concentrate the radicals (II). C. After focused ultrasound stimulus, gelatin microgels fully melt leaving behind PEG microgels and fibers (I) that are completely crosslinked resembling a continuous, bulk hydrogel. PDEGMA goes back to its original structure upon cooling (II).

FIGS. 2A-2B shows focused ultrasound responsiveness on precursor solution and granular hydrogel materials. A. An oscillatory time sweep of 20 wt % PEG-diacrylate hydrogels crosslinked via UV light shows similar stiffness compared to the FUS crosslinked gel. Heat crosslinked gels were softer than UV and FUS crosslinked gels (I; One-way ANOVA with Tukey's posthoc test, *p<0.05, ** p<0.005). Continuous hydrogel crosslinking with FUS was achieved with duty cycles as low as 33% (II). In the presence of a thermoresponsive material, PDEGMA, crosslinking time was faster than without PDGEMA (III; T-test, *** p<0.0001). B. Determination of compressive moduli using quasi-static Instron compression testing of cylindrical pucks of crosslinked granular hydrogel material (I). Crosslinking of granular hydrogel achieved from varied FUS intensities (II) and the degree of compressive stiffness increases with varied FUS duty cycles (III). One-way ANOVA with Tukey's posthoc test was conducted (*p<0.05, **p<0.01, ***p<0.001, ****p<0.0001).

FIGS. 3A-3C show material properties of FUS-crosslinked granular hydrogels. A. Confocal images of granular hydrogel pores of UV crosslinked and FUS crosslinked scaffolds (blue—fibers and red—microgels). Images taken at 10× magnification. B. Quantification of pore space in confocal images. C. Images of FUS-crosslinked and non-crosslinked granular hydrogel scaffolds both pre- and post-incubation on a rocker for 12 hours. FUS crosslinked granular materials maintain shape and structure after incubation and rocker incubation compared to microgels only and non-crosslinked granular material where it falls apart. Images show the importance of fibers for scaffold structure integrity. An unpaired T-test was conducted.

FIGS. 4A-4B show cell seeding with granular hydrogel material post-FUS and under FUS sonication. A. Schematic of HUVECs seeded on top of a FUS crosslinked granular hydrogel with a height of 2.17 mm in a transwell (I). Depth of HUVECs that travelled through the scaffold after 2 days are shown with majority of the cells at the bottom of the transwell (II). Angled (III) and side view (IV) images of HUVECs in 3D FUS crosslinked granular hydrogel via live/dead staining (Live—green, Dead—magenta) after 2 days post seeding. (scalebars=200 μm). B. Schematic of continuous (100% duty cycle) and pulsed (50% duty cycle) FUS waveforms (I). HUVECs were seeded with granular hydrogel materials and assessed for LIVE/DEAD without FUS and UV light crosslinking (II), with FUS at 100% duty cycle (III) and 50% duty cycle (IV), where majority of the cells survived within the control and 50% duty cycle condition. (scalebars=100 μm). Quantified images shows significant differences between live vs dead cells across all conditions (V). Two-way ANOVA Mixed Effects Model with Tukey's posthoc test was conducted (****p<0.0001).

FIGS. 5A-5B show Doxorubicin release from the granular hydrogel scaffolds post FUS. A. Data was normalized to the amount of drug released in the system after 48 hours. Post-FUS sonication, an initial burst release was followed by controlled release of Doxorubicin. B. Each component is assessed for release with the gelatin particle fraction's burst release accounting for a smaller fraction of the total material release in 24 and 48 hour tests. Error bars are standard deviation.

FIGS. 6A-6C show material FUS crosslinking of the granular hydrogel system in situ. A. Images of the injected granular hydrogel pre- and post-FUS stimuli. A crosslinked, solid granular hydrogel was obtained post FUS. B. Shear Wave elastography ultrasound imaging before (left) and during (right) FUS crosslinking of injected granular hydrogel system C. FUS elastography data indicate granular hydrogel stiffness is higher post-FUS compared to prior to FUS. An unpaired T-test was conducted (****p<0.0001).

FIGS. 7A-7B show (A) schematic of project overview: Two subpopulations of microgels are mixed (purple representing gelatin microgels, yellow representing PEG microgels). Upon sonication with focused ultrasound, a crosslinked granular hydrogel is obtained. (B) Co-culture environment within microgels, facilitating the emergence of microvasculature structures.

FIGS. 8A-8B show A. the schematic illustrated the setup of focused ultrasound. A precursor solution of PEG-acrylate is mixed with an initiator and placed in a heated water bath at the focal point of the ultrasound. The solution undergoes sonication at various timepoints until reaching a target temperature of 52° C. using a 1.1 MHz FUS transducer. B. Images of continuous hydrogels, showing the gelation of PEG precursor material under FUS sonication. The white opaque area in the image serves as a visual cue indicating the onset of crosslinking (I), which disappears once the gel is removed from the FUS stimuli (II).

FIG. 9 illustrates the focused ultrasound crosslinking mechanism for the granular hydrogel scaffolds. PEG microgels containing excess acrylates are mixed with PEG nanofibers containing excess norbornenes, a PEG-thiol solution, an initiator (potassium persulfate), and a thermo-responsive material (PDEGMA). All components are then centrifuged at a high rate to ensure no excess liquid remains. Upon focused ultrasound sonications at various time points, a crosslinked granular scaffold is obtained, resembling a continuous, bulk hydrogel.

FIGS. 10A-10C show A. Rheological properties were measured. Both UV and FUS crosslinking of a continuous bulk hydrogel yield similar mechanical properties. B. The inclusion of a thermoresponsive material, PDEGMA, results in a decreased crosslinking time upon FUS stimulation. Statistical analysis was conducted using GraphPad Prism, employing an unpaired T-test, ns=no significance, *** p<0.001. C. A table detailing the varied FUS duty cycles tested. A crosslinked continuous hydrogel is achieved with a low duty cycle of 33%, representing FUS being on for 0.33 ms and off for 0.67 ms.

FIG. 11 shows images of the FUS-induced crosslinked granular scaffold on a spatula after being scooped out of an Eppendorf tube (I). The crosslinked granular hydrogel scaffold retains its shape placed in excess 1X PBS solution, as indicated by the hydrogel inside red circle (II).

FIG. 12 shows images depicting gelatin microgels containing India ink (black). Initially, before FUS stimulation, gelatin microgels are fully packed in an Eppendorf tube. However, upon FUS stimulation, reaching a temperature above the target of 37° C., gelatin microgels liquidify into a solution.

FIG. 13 shows an illustration combining all components of the granular hydrogel scaffold together with a cell co-culture (HUVECs and Fibroblasts) and apply FUS sonications. Assess microvasculature formation and cell morphology.

### DETAILED DESCRIPTION OF THE INVENTION

Before the present invention is described in further detail, it is to be understood that the invention is not limited to the particular embodiments described. It is also understood that the terminology used herein is for the purpose of describing particular embodiments only and is not intended to be limiting. The scope of the present invention will be limited only by the claims. As used herein, the singular forms “a,” “an,” and “the: include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising,” “including,” or “having” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10. The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “aspect ratio” as used herein refers to a ratio between the length and diameter (or width) of a polymer molecule or a physical assembly of polymers or molecular components by processing into a nano- or microscale structure. The length and diameter (or width) of a polymer molecule or nano- or microscale structure can be measured by known analytical means and are generally understood in the art as characteristics of the shape of the molecule or nano- or microscale structure that is formed by processes including electrosprinning or molecular self-assembly. In some embodiments, the value of aspect ratio is at least 1, as the greatest measurement among all dimensions is defined as the length of the molecule or nano- or microscale structure. For example, a polymer or nano- or microscale structure with a length of 100 μm and a diameter of 2 μm has an aspect ratio of 50. Polymeric or nano- or microscale structure with a relatively high aspect ratio (e.g., 20 or greater) can be referred to as “fiber” or “fibrous.” Polymeric or fibrous structures with a relatively low aspect ratio (e.g., 5 or less) can be referred to as “microparticle” or “spherical.”

The term “hydrogel microparticles” (or HMPs) refers to hydrogel polymers or nano- or microstructures that can take various shapes, including irregular forms. While some hydrogel microparticles may be approximately spherical, other configurations are possible. The aspect ratio of spherical particles is not a limiting factor, as particle size can be all possible ranges, including non-spherical and irregularly shaped microparticles. The application of focused ultrasound (FUS) is feasible to broad ranges of hydrogel microparticles based on a mechanism-driven approach. Suitable aspect ratio of the hydrogel microparticles includes, but is not limited to, a value less than 5, such as about 1, about 1.5, about 2, about 2.5, about 3, about 3.5, about 4, or about 4.5.

The term “porosity” refers to volume percentage of void spaces in a material. For the polymer materials (such as a scaffold or a gel) used herein, the void spaces defining porosity includes all spaces inside the material that are assessable by another object, such as a molecule, a molecular complex, a particle, or a cell.

The term “crosslinked” or “crosslinking” refers to chemical reactions joining two polymers or nano- or microstructures, or two parts of a same polymer or nano- or microstructures, together through bonds that may be covalent, electrostatic, or physical interactions.

The term “stable” or “stability” as used herein in connection with a hydrogel or a polymeric material includes thermal stability over a prolonged period of time (e.g., at least 3 days) without significant (e.g., 5% or more) decomposition or loss of mechanical integrity. The term stability can also include a continued integrity at the molecular and microscale of a microparticle- or fiber-based material or system in an uncrosslinked or crosslinked state during it's designed shelf life. The shelf life of such material or system may be suitable for its intended use. For example, the material or system may be designed to degrade on a shorter time scales (e.g., a few hours to a day) for cell-delivery applications, whereas for tissue engineering applications the material or system may be designed to degrade over a longer time scales (e.g., at least a day).

The present disclosure related to methods of making and using crosslinked granular hydrogel having a desirable stability and other mechanical properties at relatively high porosity, which may be useful for a wide range of application including 3D bioprinting, regenerative medicine, and tissue engineering. Porosity in crosslinked granular hydrogels is typically increased upon decreasing the packing density. This approach is limited as low packing density reduces the inter-granule contacts that are required to create surface-surface bonds, which are critical in stabilizing the final scaffolds. While literature does not report the upper limit for porosity achieved in this fashion, porosity higher than 30-40% have not been reported using this approach, and none have been cultured for longer than 7 days. Further, in vivo studies have not reported granular hydrogel scaffolds with porosity higher than 20% but have shown beneficial effects toward cellular infiltration as porosity and pore size increased. There is a need for methods to yield structurally more permissive materials, such as materials which have large amounts of porosity and/or contain components that can move past one another within a stable bulk material.

As used herein, FUS refers to a non-invasive technique that employs focused beams of ultrasound energy to deliver acoustic energy to a target location, wherein multiple ultrasound waves converge to produce precise thermal and/or mechanical effects at the focal point. FUS may be applied continuously or in pulsed modes and may be guided by magnetic resonance imaging (MRI) or ultrasound imaging for precise targeting.

The present disclosure addresses this need. In various embodiments, the present disclosure provides crosslinked granular hydrogel compositions and methods of preparing and/or using a crosslinked granular hydrogel. In one aspect, the present disclosure provides a method of preparing a crosslinked granular hydrogel, which comprises: (i) mixing: a hydrogel microparticle having a first crosslinking group, a polymeric fiber having a second crosslinking group, an initiator, and a crosslinker to form a precursor composition; and (ii) applying focused ultrasound (FUS) to the precursor composition, whereby each of the first crosslinking group and the second crosslinking group reacts with the crosslinker, thereby the hydrogel microparticle and the polymeric fiber are crosslinked to form the granular hydrogel.

In some embodiments, the precursor composition may further comprise a removable particle, which upon removal changes the porosity of the granular hydrogel, and/or a viscosity promotor, which increases viscosity upon FUS-induced heating. In some embodiments, the method may further comprise removing the removable particle, thereby changing the porosity of the granular hydrogel. For example, the crosslinked granular hydrogel further comprises removable particles and have a porosity of at least 10%, at least 20%, at least 30%, at least 40%, at least 50%, at least 60%, at least 70%, at least 80%, or at least 90%. Upon removal of the removable particles, the porosity can increase to, for example, at least 10%, at least 20%, at least 30%, at least 40%, at least 50%, at least 60%, at least 70%, at least 80%, or at least 90%. Remarkably, the scaffold can be stable and have desirable mechanical properties at a porosity of at least 30%.

In some embodiments, the removable particle comprises gelatin, extracellular matrix-derived hydrogel particles (including Matrigel), polymethylmethacrylate particles, poly alpha esters, poly ester amides, particles formed from hydrogels with enzymatically cleavable crosslinks, particles from hydrogels with physical crosslinks, alginate particles, agarose particles, pluronic, poly-NIPAAM-based particles, or combination thereof. In some embodiments, the removable particles comprise gelatin.

The polymeric fibers, the hydrogel microparticles, and the removable particles can form a stable structure. Removal of the particles can increase the void space between the polymeric fibers and the hydrogel microparticles, thereby increasing the porosity of the granular hydrogel. The removable particles can be removed by known procedures, such as thermal melting, backwash, enzymatic degradation, hydrolysis, chemical or physical disruption of physical bonds (e.g., through solvents, changes in ionic charges, changes in hydrophobicity/hydrophilicity within the removable particles, the use of solutes with affinity for components of the crosslinking scheme) within the removable particles, and any other form or removal procedure of sacrificial particles.

In some embodiments, the viscosity promotor comprises poly(di(ethylene glycol) methyl ether methacrylate (PDEGMA). Other exemplary viscosity promoters include, but are not limited to, poly(N-isopropylacrylamide).

In some embodiments, the precursor composition further comprises gelatin and poly(di(ethylene glycol) methyl ether methacrylate (PDEGMA).

The first and/or second crosslinking group can comprise a C═C group or a thiol group. In some embodiments, the crosslinking group comprises a photoinitiated crosslinker. In some embodiments, the crosslinking groups comprise norbornene, vinylacetyl, vinyl ester, vinylsulfonyl, vinyl ether, allyl, acrylate ester, methacrylate ester, acrylamido, malcimido, propenyl ether, allyl ether, alkenyl, unsaturated ester, dienyl, methacrylate, acrylate, vinyl sulfone, azide, cyclooctyne, hydrazide, aldehyde, thrombin, fibrin, thiols, and combinations thereof. In some embodiments, the crosslinking groups comprise norbornene, methacrylate, thiol, or a combination thereof. Other suitable crosslinking groups known in the art also can be used. In some embodiments, each of the first each of the first crosslinking group and the second crosslinking group is independently norbornene, methacrylate, acrylate, vinyl sulfone, azide, cyclooctyne, hydrazide, aldehyde, thrombin, fibrin, or combination thereof.

Suitable crosslinkers include, but are not limited to crosslinkers comprising thiol groups. For example, the crosslinker can be PEG-thiol. In some embodiments, each of the first each of the first crosslinking group and the second crosslinking group is independently norbornene or acrylate, and the crosslinker comprises thiol groups.

The hydrogel microparticle and/or polymeric fiber as used herein may comprise a hyaluronic acid; a poly(ethylene glycol) (PEG); a polynorbornene; heparin; a polysialic acid; a poly(glycerol); a poly(oxazoline); a poly(vinylpyrrolidone); a poly(acrylamide); a poly(N,N-dimethylacrylamide); a poly(acrylamide); a poly(lactic acid) (PLA); a polyglycolide (PGA); a copolymer of PLA and PGA (PLGA); a poly(vinyl alcohol) (PVA); poly(ethylene oxide); a poly(ethylene oxide)-co-poly(propylene oxide) block copolymer; a poloxamine; a polyanhydride; a polyorthoester; a poly(hydroxy acids); a polydioxanone; a polycarbonate; a polyaminocarbonate; a poly(vinyl pyrrolidone); a poly(ethyl oxazoline); a polyurethane; a carboxymethyl cellulose; a hydroxyalkylated cellulose; a polypeptide; a polypeptoid; a polysaccharide; a carbohydrate; collagen; a extracellular matrix-derived hydrogel; gelatin; alginate; dextran; self-assembled structures including self-assembled peptides or self-assembled peptide amphiphiles; or combinations thereof. In some embodiments, the hydrogel microparticle comprises poly(ethylene glycol) (PEG). In some embodiments, the polymeric fiber comprises poly(ethylene glycol) (PEG).

In the present crosslinked granular hydrogel, the polymeric fibers and the hydrogel microparticles can form a stable network. For example, the polymeric fibers and the hydrogel microparticles can form a reinforced structure with improved stability and mechanical properties, compared to a system that includes the polymeric fibers alone or the hydrogel microparticles alone, particularly where the hydrogel has high porosity (e.g., at least 10%). The polymeric fibers can have a suitable aspect ratio, which includes, but is not limited to, a value of at least 5. In some embodiments, the polymeric fibers of the hydrogel have an aspect ratio of at least 15. The aspect ratio of the polymeric fibers of the hydrogel can be at least 20, at least 25, at least 30, at least 50, at least 100, at least 200, or at least 300. In some embodiments, the polymeric fibers of the hydrogel have an aspect ratio of at least 25.

In some embodiments, the polymeric fibers of the crosslinked granular hydrogel are electrospun fibers. The polymeric fibers can be produced by known electrospinning processes. As a non-limiting example, the polymers (e.g., norbornene-containing polymers, thiol-containing polymers, polyethylene oxide) can be suspended in an aqueous solution, and the solution was electrospun to produce a polymeric fiber. The preparation method can further comprise centrifugation to isolate the electrospun fiber. In some embodiments, at least 50% v/v of the electrospun fiber is recovered from centrifugation. In some embodiments, the polymeric fibers have an aspect ratio of at least 30. Under these conditions, the resulting crosslinked granular hydrogel can maintain stability and desirable mechanical properties at a wide range of porosity, such as 30%-90%.

In some embodiments, the polymeric fibers of the crosslinked granular hydrogel and/or scaffold have a mean diameter of about 0.3 μm to about 7 μm. The mean diameter can be, for example, about 0.3 μm to about 6 μm, about 0.3 μm to about 5 μm, about 0.3 μm to about 4 μm, about 0.3 μm to about 3 μm, about 0.5 μm to about 2.5 μm, about 0.5 μm to about 2.0 μm, or about 0.5 μm to about 1.5 μm. In some embodiments, the mean diameter is about 0.5 μm to about 2.5 μm, including but not limited to, about 0.6 μm, about 0.7 μm, about 0.8 μm, about 0.9 μm, about 1.0 μm, about 1.1 μm, about 1.2 μm, about 1.3 μm, about 1.4 μm, about 1.5 μm, about 1.6 μm, about 1.7 μm, about 1.8 μm, about 1.9 μm, about 2.0 μm, about 2.1 μm, about 2.2 μm, about 2.3 μm, and about 2.4 μm.

In some embodiments, the polymeric fibers of the crosslinked granular hydrogel and/or scaffold have a mean length of about 35 μm to about 1 cm. The mean length can be, for example, about 35 μm to about 8 mm, about 35 μm to about 6 mm, about 35 μm to about 4 mm, about 35 μm to about 2 mm, about 35 μm to about 1 mm, about 35 μm to about 800 μm, about 50 μm to about 800 μm, about 50 μm to about 600 μm, about 50 μm to about 400 μm, or about 50 μm to about 200 μm. In some embodiments, the mean length is about 50 μm to about 200 μm, including but not limited to, about 60 μm, about 70 μm, about 80 μm, about 90 μm, about 100 μm, about 120 μm, about 130 μm, about 140 μm, about 150 μm, about 160 μm, about 170 μm, about 180 μm, and about 190 μm.

In some embodiments, focused ultrasound (FUS) parameters can be modulated to target hydrogels with specific mechanical properties. In one embodiment, FUS is applied with a continuous duty cycle of 100%, ensuring uninterrupted energy delivery to the target volume. The intensity of FUS can be adjusted through input voltage settings to achieve desired mechanical properties in the resulting hydrogels. In other embodiments, FUS applies 90% cycle duty, a 75% cycle duty, a 50% duty cycle, a 33% duty cycle, a 25% cycle duty, or a 10% cycle duty.

In an embodiment, FUS is applied at an intensity of 8 W/cm² while maintaining a 100% duty cycle. In another embodiment, FUS is applied at an intensity of 12 W/cm² while maintaining the 100% duty cycle. In another embodiment, FUS is applied at an intensity of 18 W/cm² while maintaining the 100% duty cycle. The resulting hydrogels of these methods have a compressive moduli of about 5 kPa, about 10 kPa, about 15 kPa, or about 20 kPa. Additional parameters that may be varied in different embodiments include the duration of FUS application, focal point depth, transducer frequency, and cooling conditions. These parameters may be adjusted in combination with intensity settings to further fine-tune the properties of the resulting hydrogels for specific applications.

In some embodiments, the hydrogel microparticles is in the form of a 1:1 suspension or solution in PBS. The proportion of each can be increased or decreased as needed. The polymeric fibers can be in the form of 1:9 suspension or solution in PBS. The amount of polymeric fibers is flexible and can be adjusted to match the volume of hydrogel microparticles. In some embodiments, the ratio of hydrogel microparticles to polymeric fibers is 1:1, 2:1, 3:1, 4:1, 5:1, 6:1, 7:1, 8:1, 9:1, or 10:1. For example, 300 μL of the hydrogel microparticles (1:1 in PBS) can be used with polymeric fibers (1:9 in PBS), with a final concentration of the polymeric fibers at 5% (v/v).

In some embodiment, a removable particle (e.g., gelatin) is present in the form of a 1:1 suspension or solution in PBS, and amount (e.g., 100 μL) can be adjusted according to the amounts of the hydrogel microparticles and polymeric fibers. In some embodiments, the ratio of hydrogel microparticles to removable particles is 1:1, 2:1, 3:1, 4:1, 5:1, 6:1, 7:1, 8:1, 9:1, or 10:1.

In some embodiments, the crosslinker has a stock concentration of 10% (w/v), and can be used to reach a final concentration of 0.7% (w/v) in the precursor composition. In some embodiments, the crosslinker concentration can be 15% (w/v), 20% (w/v), 30% (w/v), 40% (w/v), 50% (w/v), 60% (w/v), 70% (w/v), 80% (w/v), or even 90% (w/v) in the precursor composition.

The radical initiator, in some embodiments, is 1.5% (w/v) making up 0.2% of the final concentration. In other embodiments, the radical initiator makes up 0.2%, 0.4%, 0.5%, 0.6%, 0.7%, 0.8%, 0.9%, 1.0%, 1.5%, 2.0%, 2.5%, 3.0%, 4.0%, 5.0%, 10%, 20%, 30%, 40%, or 50% of the final concentration (w/v). Additionally, any suitable photoinitiator or thermoinitiator can be used in the present method. Exemplary initiators include APS, LAP, I2959, and EosinY.

In some embodiments, the viscosity promoter is 2.27% (w/v) in the stock making up 0.16% of the final concentration. In some embodiments, the viscosity promoter is about 1%, 2%, 3%, 4%, 5%, 6%, 7%, 8%, 9%, 10%, 20%, 30%, 40%, or 50% of the final concentration. This may be adjusted based on specific experimental conditions. These adaptable ranges ensure that the system remains versatile and customizable for various applications.

In some embodiments, the crosslinked granular hydrogel contains about 5% to about 100% by volume polymeric fibers. The volume percent can be calculated using known techniques and systems, for example, based on the interstitial volume and the packing density of the scaffold. The volume percent of the polymeric fibers can be about 10% to about 80%, such as about 10%, about 20%, about 30%, about 40%, about 50%, about 60%, about 70%, or about 80%. Addition of liquid or a removable particle (e.g., a particle that is removed by thermal melting) can lower the vol % of the fibers. For example, an 80 vol % fiber system can include, by volume, 8 parts of fibers and 2 parts of medium; or 8 parts of fibers, 1 part of gelatin particles, and 1 part of medium; or 8 parts of fibers and 2 parts of particles. It is understood that some degree of porosity would remain between the fibers, as the particles theoretically could not perfectly fill all voids. In other words, actual void space between particles would not quite reach 0% as the fiber approaches 100% by volume in the material

The present method may produce an injectable precursor composition in step (i). In some embodiments, the method further comprises: (i-a) injecting the precursor composition from (i) into a subject; and (ii) applying focused ultrasound (FUS) to the injected precursor composition in the subject, thereby forming a crosslinked granular hydrogel in the subject. Thus, the present method can be used for delivering a cell. In some embodiments, at least one of the hydrogel microparticle and the polymeric fiber in the precursor composition comprises an embedded cell.

The present method can also be used for drug delivery. In some embodiments, at least one of the hydrogel microparticle and the polymeric fiber in the precursor composition comprises an embedded drug. In some embodiments, the precursor composition further comprises a removable particle, wherein the removable particle is embedded with a first portion of the drug, and wherein at least one of the hydrogel microparticle and the polymeric fiber is embedded with a second portion of the drug.

The injectable precursor composition can be administered subcutaneously. In particular embodiments, step (i-a) comprises subcutaneous injection of the precursor composition from (i) into a subject, e.g., to deliver a cell or a drug.

In some embodiments, the methods described herein occurs in vivo. The in vivo application of this hydrogel system comprises injecting the precursor composition into a subject; and applying focused ultrasound (FUS) to the injected precursor composition in the subject, thereby forming a crosslinked granular hydrogel in the subject. The success of the in vivo formation of the crosslinked granular hydrogel depends on careful control of these parameters, along with continuous assessment of both local and systemic responses to ensure safety and efficacy of the procedure. In some embodiments, at least one of the hydrogel microparticle and the polymeric fiber in the precursor composition comprises an embedded cell. In some embodiments, this method is used to enhance healing processes by promoting cellular integration and tissue remodeling, making it an effective strategy in regenerative medicine. In further embodiments, at least one of the hydrogel microparticle and the polymeric fiber in the precursor composition comprises an embedded drug. In some embodiments, the method has the potential to efficiently encapsulate and deliver a wide range of therapeutics, including biologics, small molecules, proteins, and hormones.

In some embodiments, the precursor composition further comprises a removable particle, wherein the removable particle is embedded with a first portion of the drug, and wherein at least one of the hydrogel microparticle and the polymeric fiber is embedded with a second portion of the drug. In some embodiments, the method is used for multi-phase drug release by utilizing microgels with adjustable release profiles. While it is effective for two-phase drug delivery, it also allows for additional phases by simply modifying the composition of the microgels. In some embodiments, the in vivo injection of the precursor composition comprises subcutaneous injection. In some embodiments, the method can be used for various delivery mechanisms, including catheter-based administration for targeted drug release in the heart or other organs.

In some embodiments, the subject is a human. In other embodiments, the subjects are laboratory mice, rats, rabbits, guinea pigs, sheep, goats, pigs, dogs, cats, non-human primates, horses, cattle, zebrafish, and other experimental animal models.

In another aspect, the present disclosure provides a method of regenerating a tissue in a subject. The method includes preparing a crosslinked granular hydrogel from a precursor composition injected into the subject, the precursor composition having an embedded cell, according to the process described herein; and allowing the cell to grow in the subject, thereby regenerating a tissue from the cell. In some embodiments, the method is used for tissue regeneration in a human.

In another aspect, the present disclosure provides a method of delivering a drug to a subject. The method includes preparing a crosslinked granular hydrogel from a precursor composition injected into the subject, the precursor composition having the drug embedded therein, according to the process described herein; and releasing the drug from the crosslinked granular hydrogel, thereby delivering the drug to the subject. In some embodiments, the precursor composition further comprises a removable particle. The removable particle is embedded with a first portion of the drug, and at least one of the hydrogel microparticle and the polymeric fiber is embedded with a second portion of the drug. In some embodiments, the method is used for delivering a drug to a human.

In another aspect, the present disclosure provides a crosslinked granular hydrogel produced by the method described herein. In another aspect, the present disclosure also provides a scaffold comprising the crosslinked granular hydrogel produced by the methods described herein.

In some embodiments, the polymeric fibers and/or the hydrogel microparticles of the scaffold are crosslinked. For example, a fiber polymer molecule can be crosslinked with one or more other fiber polymer molecules, a fiber polymer molecule can be crosslinked with one or more hydrogel microparticles, or a hydrogel microparticle can be crosslinked with one or more other hydrogel microparticles. The crosslinking may improve the stability of the scaffold or adjust the mechanical properties of the scaffold. In some embodiments, the crosslinking of the fibers (or hydrogel microparticles) of the scaffold is understood to be the crosslinking between a fiber (or a hydrogel microparticle) with another fiber or a hydrogel microparticle, which is separate and different from the process of making the fibers (or hydrogel microparticles) involving crosslinking polymer molecules as building blocks of the fibers (or hydrogel microparticles).

In another aspect, the present disclosure provides a method of culturing cells. The method comprises: mixing the cells with the scaffold as described herein, thereby the cells are embedded in the scaffold; and culturing the cells embedded in the scaffold. In some embodiments, the crosslinked granular hydrogel and/or scaffold comprises a stable network formed by the polymeric fibers (e.g., having an aspect ratio of at least 25) and hydrogel microparticles. In some embodiments, the cell comprises cells critical for vasculature formation including human umbilical vein endothelial, murine myoblasts (C2C12s, ATCC, etc), fibroblasts, induced pluripotent stem cells, mesenchymal stromal cells, neural stem cells, hepatoctyes, macrophages, beta cells and pancreatic islets, cells of the immune system, cells of any tissue or organ system in the body, cells derived from diseased tissue including cancer and tumor cells, genetically modified cells, and any other cell that can be cultured in 2D or isolated from an organism for 3D culture.

The stability and mechanical properties of the scaffold can be adjusted by crosslinking. However, due to the long-range interactions of the polymeric fibers as described herein. The degree of crosslinking may be controlled using known techniques to adjust the stability of the scaffold for specific applications in cell culture and tissue engineering. Crosslinking, if desired, can be carried out before or after mixing the cells with the scaffold. The use of the present scaffold for applications in regenerative medicine and tissue engineering may involve known bioprinting techniques. In some embodiments, the present method may further comprise, prior to culturing the cells embedded in the scaffold, extruding the scaffold having embedded cells. In some embodiments, the scaffold with embedded cells has suitable mechanical properties to form an extrudable composition, such as a printable or injectable composition. As a nonlimiting example, the scaffold having embedded cells can be extruded by 3D printing. Suitable 3D printing systems (including printers and software) include those known in applications of bioprinting and tissue engineering. In some embodiments, the scaffold is used as a support material (also known as a support bath) into which materials and cells might be printed (in 3D printing approaches known as embedded printing). In some embodiments, the scaffold as a support material includes cells, fibers, microparticles, and removable particles in various ratios, as desired. In some embodiments, the present scaffold is used as both a support material and an extruded material.

Cell growth and cell behavior can be affected by the porosity of the scaffold as described herein, which in turn can be controlled by inclusion of removable particles. Removal of such particles can increase the porosity of scaffold, thereby changing the microenvironment in which the cells are cultured. In some embodiments, the scaffold comprises removable particles and the method further comprises, prior to mixing the cells with the scaffold, removing the removable particles, thereby changing the porosity of the scaffold.

In another aspect, the present disclosure also provides an implant comprising a crosslinked granular hydrogel produced from an injected precursor composition as described herein. In some embodiments, the injected precursor composition comprises an embedded cell. In further embodiments, the injected precursor composition comprises an embedded drug.

### EXAMPLES

### Example 1

Tissue regeneration depends on material properties with porosity supporting revascularization and tissue regrowth, and there are significant challenges in creating injectable porous hydrogels that can be crosslinked deep within tissues. To address this challenge, the present study reports the design and characterization of a highly porous granular hydrogel system that leverages the unique strengths of deeply penetrating, minimally invasive focused ultrasound (FUS). Specifically, the present study developed a composite granular hydrogel scaffold composed of polyethylene glycol (PEG)-based microgel particles, fibers, and pore-defining gelatin microgels. Upon applying FUS, crosslinking of the PEG components stabilized the bulk granular hydrogel, while melting of the gelatin microgel imbued porosity. FUS parameters were determined that crosslinked scaffolds both in vitro, while maintaining cytocompatibility, and after subcutaneous injection in mouse cadavers. FUS crosslinked granular hydrogels exhibited fiber-dependent stability and viscoelastic properties comparable to photo-crosslinked granular hydrogels. Overall, this work defines a FUS-responsive granular system and extends the potential of FUS as a novel, non-invasive method for crosslinking regenerative hydrogels.

Hydrogels are well established in regenerative medicine due to their properties that closely mimic natural tissues and potential for application-specific design. Characterized by both a high water content and a three-dimensional (3D) network structure, hydrogels are similar to the extracellular matrix (ECM) in human tissues. Additionally, their mechanical properties and degradation rates can be precisely engineered to match target tissue properties and promote regeneration. Hydrogels have been used in diverse applications in wound healing^{1,2}, cartilage repair^{3,4}, bone regeneration, cardiac tissue injuries⁵, and neural tissue regeneration⁶. In many medical contexts, injection of a liquid hydrogel, followed by a rapid transition to a gel state, is desirable. To enable the delivery of hydrogel precursor solution and the formation of a stable nanoporous network at the site of injection, rapid hydrogel crosslinking is required. Hydrogels have been engineered to crosslink through mechanisms such as matching crosslinking kinetics to timing of delivery, mixing during delivery, pH changes, electrostatic interactions, or enzymatic reactions⁷⁻¹⁰. However, achieving precise gelation after injection remains challenging, as the aforementioned methods lack spatiotemporal control over crosslinking. Externally triggered crosslinking via photoinitiation provides some access to controlling the onset of crosslinking, but light has a limited penetration depth^{11,12}, leaving the need for cross-linking within deep tissue unresolved.

Ultrasound technologies offer a direct, controllable, and non-invasive stimulus^{13,14} that can alter structural characteristics of hydrogels and trigger crosslinking^{15 16-21}. These acoustic waves have been shown to polymerize hydrogel formulations¹⁶⁻²¹ and modulate the porosity of engineered scaffolds²¹⁻²³. Through mechanisms like acoustic cavitation, which generates instantaneous large pressure changes and high temperatures, ultrasound can initiate radical reactions, even in the absence of initiator, and restructure material architecture. Focused ultrasound (FUS), which entails concentrating acoustic energy into a very small ellipsoidal volume, is a means of externally triggering minimally invasive hydrogel crosslinking within minutes via generation of radicals^{19,24,25} with spatial and temporal control²⁵⁻²⁹. In essence, FUS can be applied to create a small area of intense energy without affecting or heating tissue along the beam path^{16,27}. FUS can also initiate dynamic changes in thermally responsive biomaterials^{20,30} that change conformation based on temperature, for example poly(N-isopropyl acrylamide) and poly(oligoethylene glycol methacrylate) s that assume a globule form in heated environments and coiled conformation in cooler temperatures. Despite these advantages, challenges remain when crosslinking polymer solutions. Hydrogel precursor solutions may flow away from the target site before polymerization is complete, and after crosslinking, form nanoscale porous structures that are often not conducive to supporting tissue regrowth and vascularization.

Granular hydrogel systems address key limitations of bulk hydrogels by offering injectability in conjunction with stable microporous structures that support tissue integration and nutrient transport. These systems, composed of discrete hydrogel microgels, flow during injection and stabilize post-delivery through physical jamming and interparticle crosslinking (annealing). In regenerative medicine, microporous annealed particle (MAP) hydrogels have enhanced tissue regrowth¹⁵ and demonstrated favorable immunological responses³¹. Their modularity allows further tuning: inclusion of electrospun hydrogel fibers improves mechanical stability and supports mesoscale (100 μm-1 mm) pores³², whereas inclusion of removable gelatin particles allows engineering porosity into the scaffolds to facilitate infiltration of cells and nutrients. By adjusting the ratio of removable to non-removable components, interconnected pores can be formed without the compaction issues that arise in diluted systems during injection³³. Moreover, long-range particle-fiber interactions help maintain pore structure over time and support high cellularity³².

FUS has been used to trigger chemical reactions in crosslinking free flowing inks for 3D printing^{25,34} and to break bonds within dynamic crosslinked hydrogels for protein delivery^{17,20,22,35}. However, the use of FUS to crosslink granular hydrogel scaffolds and use for controlled drug release in these system remains unexplored. In this work, it was demonstrated that FUS successfully crosslinks granular hydrogel materials in situ with the incorporation of hydrogel fibers, and that the resulting materials are highly porous, toward ultimately supporting wound healing and tissue regeneration. Heating at the FUS focal point melted the gelatin microgels and induced crosslinking of the microgels and fibers in vitro. To evaluate this system's potential for use in vivo, the hydrogel formulation was injected subcutaneously into a mouse cadaver model and observed that FUS induced crosslinking and generated stable fiber-reinforced hydrogels localized to subcutaneous injection sites. This approach highlights the potential of FUS to be used in conjunction with granular hydrogels for translational applications and therapeutic purposes.

### Designing FUS-Responsive Granular Hydrogels

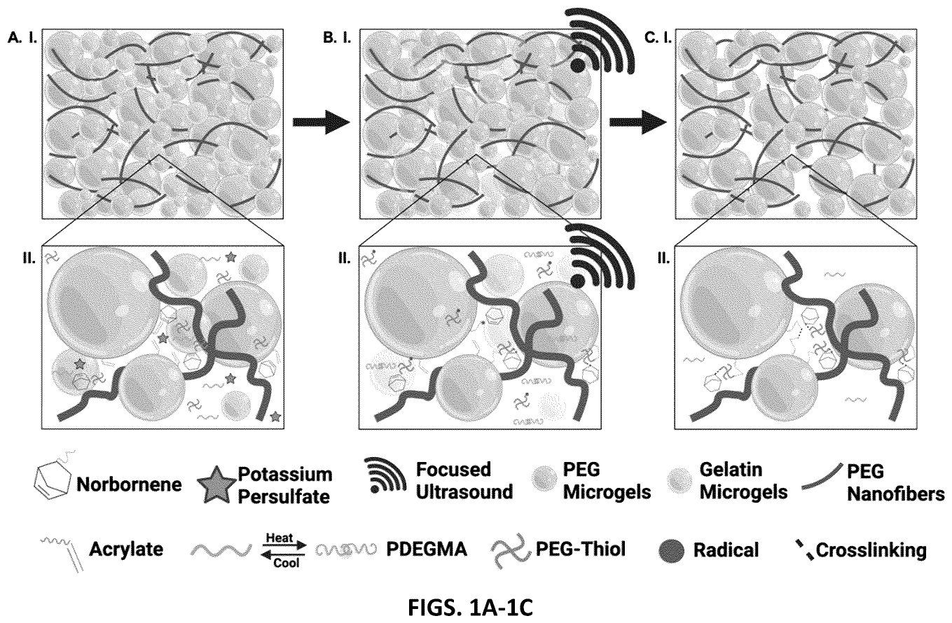

The injectable granular hydrogel formulations include PEG microgels with excess acrylates, PEG fibers with excess norbornenes, and gelatin microgels (FIG. 1A). In this system, PEG microgels and fibers were designed to comprise the final structure of granular hydrogels, with gelatin microgels as a removable component for engineering porosity. The inclusion of the gelatin microgels, rather than simply diluting the PEG components, allows precise control over the final gel porosity by preventing the loss of void space that can occur during injection when using dilution-based methods. To form crosslinks between the acrylate-and norbornene-functionalized PEG microgels and fibers upon FUS sonication, soluble tetra PEG-thiol was included. It was hypothesized that FUS sonication, in the presence of the free radical initiator potassium persulfate (KPS), which generates radicals in response to both light-and thermal-based cues and poly(diethylene glycol methyl ether methacrylate) (PDEGMA), a thermally responsive acoustic absorber material³² intended to reduce convective motion upon heating, would support interparticle crosslinking (FIG. 1B). Specifically, it was hypothesized that radicals generated by FUS³⁶ would initiate thiol-ene chemical reactions near the focal point, forming crosslinks between the acrylate-functionalized microgel and norbornene-functionalize fiber components through the soluble tetra PEG-thiol molecules (FIG. 1B, II). Following a recently reported strategy to assist the cross-linking reaction by locally concentrating ultrasound-generated radicals²⁵, the present study included a thermally responsive polymer. Since PDEGMA undergoes a coil to globule transition upon heating, it was hypothesized that FUS-induced heating would trigger this transition, resulting in locally increased viscosity. Simultaneously, upon applying FUS, gelatin microgels (melting point: 37° C.) were expected to liquefy (FIG. 1B). After FUS application, a crosslinked granular hydrogel will remain, containing pores formed by gelatin microgel melting (FIG. 1C).

Crosslinking PEG Hydrogel Precursors and PEG Granular Hydrogels with FUS

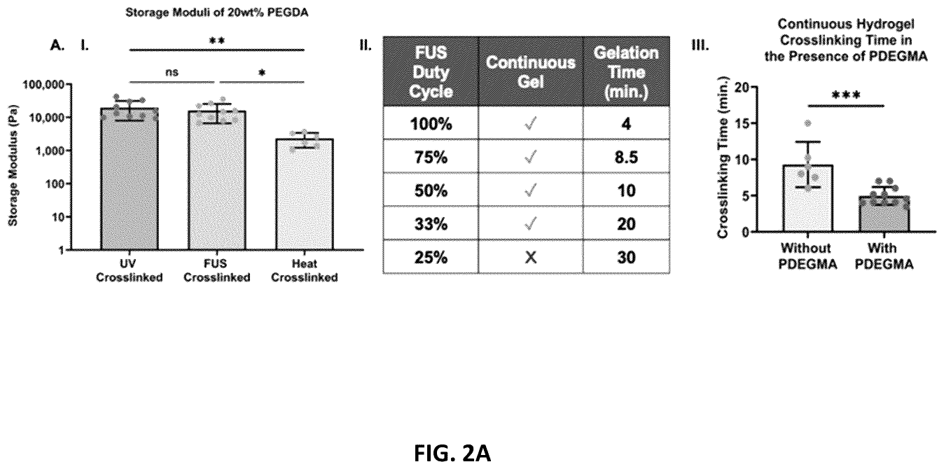

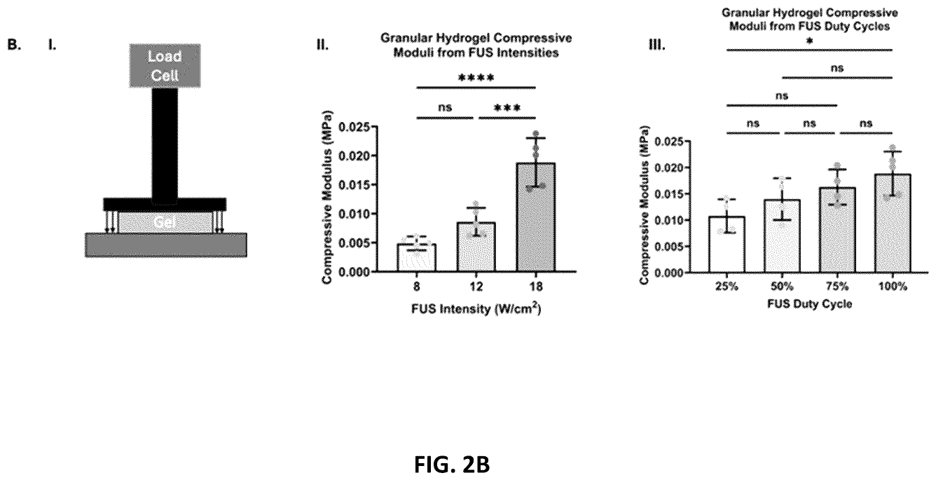

To determine the FUS parameters for interparticle crosslinking, crosslinking of a continuous, bulk hydrogel was first evaluated before moving to a granular formulation, where a lack of stabilization via annealing crosslinking might not reflect non-reactivity. Continuous PEG hydrogels have been crosslinked using ultrasound^{37,38}. A hydrogel precursor solution containing 20 wt % PEG-diacrylate (PEGDA)^{16,17}, KPS as a radical generator, and PDEGMA was prepared as a thermoresponsive acoustic absorber. Samples were sonicated in microcentrifuge tubes with FUS, and gelation was observed within 10 minutes. As a confirmation of gelation, the resulting hydrogels were determined to have storage moduli of ˜10 kPa as measured by rheology (FIG. 2A, I). To confirm crosslinking via FUS was similar to established photocrosslinking, hydrogels formed via FUS were compared to hydrogels formed form 20 wt % PEGDA and 10 mM lithium phenyl-2,4,6-trimethylbenzoylphosphinate (LAP) using photocrosslinking. Photocrosslinked gels had a similar stiffnesses of ˜11 kPa, providing evidence that FUS applied with the chosen parameters could drive crosslinking to the same level as achieved with photocrosslinking.

Further studies were conducted to determine whether the crosslinking observed was caused by KPS responding to heat alone, or if other effects of FUS, like mechanical effects also triggered crosslinking through KPS³⁹. KPS was used to crosslink the present hydrogel precursor thermally. The resulting PEGDA gel exhibited a stiffness of only ˜1.5 kPa (FIG. 2A, I). The significant decrease in stiffness observed when using only a purely thermal stimulus, similar to the heating component of FUS, indicates FUS crosslinking is also due to non-thermal mechanism(s), energy to the crosslinking observed when using FUS to trigger radical generation and acrylate polymerization in the precursor solution.

Additional experiments were conducted to understand how FUS duty cycle, which is the percent of total treatment time in which ultrasound waves are applied, influences in the extent of crosslinking. To this end, FUS was applied with continuous (100% duty cycle) and pulsed waveforms (10 ms burst) with burst timing corresponding to 75%, 50%, 33%, and 25% duty cycles. A continuous, bulk PEGDA hydrogel was obtained with duty cycles as low as 33% duty cycle, but gelation was not observed with a 25% duty cycle (FIG. 2A, II). Decreased duty cycles corresponded to longer gelation times.

To test the importance of including an acoustic absorber, PDEGMA, in the system, FUS crosslinking of continuous hydrogel precursor solutions with and without PDEGMA were compared. The reults first verified that PDEGMA was undergoing the intended thermally-induced coil-to-globule transition in the hydrogel precursor solutions: indeed, heating the solution rendered the initially clear precursor solution turbid, indicative of globule formation that should increase viscosity. In the absence of PDEGMA, 2-3× longer times (10-12 min) were required to crosslink a continuous PEGDA hydrogel (FIG. 2A, III), showing the effect of PDEGMA on gelation.

Further experiments were conducted to test how FUS crosslinking affects hydrogel stiffness as a function of FUS intensity. After FUS sonication, samples underwent compressive mechanical testing via Instron (FIG. 2B, I). FUS intensity was modulated while maintaining a 100% continuous FUS duty cycle. Input voltages of 8 W/cm², 12 W/cm², and 18 W/cm² yielded stable gels with compressive moduli of 5 kPa, 10 kPa, and 20 kPa, respectively (FIG. 2B, II), indicating FUS intensity correlates with granular hydrogel stiffness.

Building on observations from continuous hydrogels, the present study next investigated whether altering the FUS duty cycle, while keeping total treatment time constant, would affect the compressive modulus of crosslinked granular hydrogels (FIG. 2B, III). The results indicate a modest trend in increasing moduli with duty cycle, suggesting increasing interparticle crosslinking within the system as duty cycle increased. Indeed, a 25% duty cycle yielded a stable granular hydrogel with a stiffness of 10 kPa compared to the continuous, 100% duty cycle FUS waveform that resulted in granular hydrogels with 19 kPa stiffnesses. A statistically significant difference in granular hydrogel stiffness is only observed between the 25% and 100% duty cycle groups. Because duty cycle decreases result in lower time-averaged temperatures⁴⁰. A stable hydrogel with ˜12 kPa compressive moduli in the 50% duty cycle group was obtained, where the focal point temperature only increased to 45° C. for the 15 minutes of FUS exposure. This suggests that the crosslinking of the granular material is at least partially due to radical production through interactions of KPS with the FUS energy as the material properties are similar to continuous waveforms (100% duty cycle) where the focal point temperature increases to 55° C. While previous studies have demonstrated hydrogel crosslinking with focused ultrasound⁴⁰, they often rely on high power levels and subsequent substantial heating to induce crosslinking. While useful for 3D printing applications, this approach poses a risk in vivo, especially near thermally sensitive structures such as vasculature and nerves, making the present low-power approach particularly advantageous for safer and more controlled in situ crosslinking. Future optimization of duty cycle and ultrasound intensity will enable crosslinking of gel in vivo without significant risk to collateral tissue.

### FUS-Crosslinked, Fiber-Enhanced Granular Hydrogels Porosity and Stability

Having confirmed the formation of mechanically robust fiber-reinforced granular hydrogels with FUS-induced crosslinking, the present study next considered the resulting porous microstructures, the effects of the fibers on scaffold stability, and cellular integration into pore space in vitro. Ratiometric incorporation of meltable gelatin microgels among the particle and fiber components allows specification of the pore fraction³². To confirm that FUS melts gelatin microgels, the effects of FUS was tested on gelatin microgels containing a black dye in a microcentrifuge tube for visualization. Before application of FUS, these gelatin microgels demonstrated solid-like properties as a packed, granular hydrogel within a microcentrifuge tube that did not flow during handling. Immediately after FUS stimuli, the granular hydrogel melted into a continuous, flowing fluid.

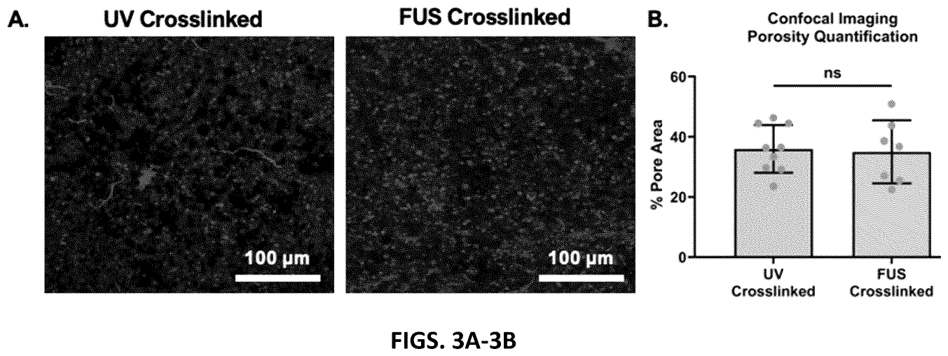

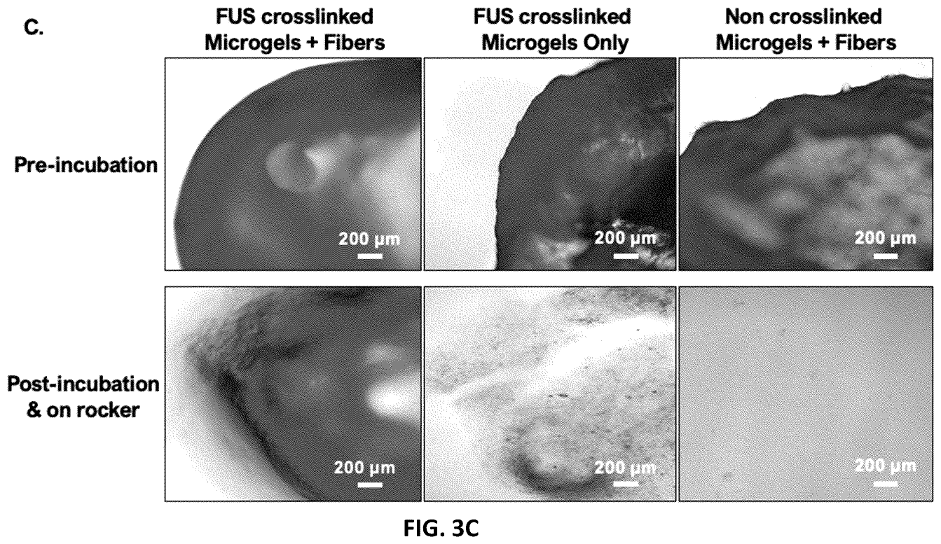

Adding the fibrous components to the gelatin microgels sustain the highly porous microstructure that emerges when gelatin melts³². To visualize the resulting porosity, FUS-crosslinked and photocrosslinked gels were imaged by confocal imaging (FIGS. 3A-B). In these experiments, crosslinking via FUS and UV light were tested, with the UV-crosslinked samples serving as a control where crosslinking occurred without heating. Samples placed under UV light were subsequently incubated at 37° C. to melt gelatin microgels. To visualize pore space via confocal imaging, PEG microgels fluorescently tagged with thiolated rhodamine-B and PEG fibers tagged with 5(6)-carboxyfluorescein amidite (FAM) were formulated. Again, samples crosslinked by FUS were compared to photocrosslinked samples. Microgel (red) and fiber (pseudocolored blue) distributions appear similar (FIG. 3A). Quantification of unlabeled pore spaces in confocal images show similar porosity area coverages (FIG. 3B). This suggests that when scaffolds remain continuously hydrated, their material architectures remain similar independent of the crosslinking mechanism. Scanning electron microscopic images of these scaffolds show that the FUS crosslinked gel exhibits larger pores compared to UV crosslinked gel. Quantification of porosity in SEM micrographs shows that pores within the FUS crosslinked granular hydrogels contribute to ˜30-40% of the total area seen, compared to 20-30% in the photocrosslinked materials. The slight increase of pore space, as quantified using SEM, may be due to the effects of ultrasonic treatment, which, at higher powers (>40W) than used here (8-12 W), has been shown to cause increases in the mean pore size, pore interconnectivity, and porosity in tissue scaffolds⁴¹

To further illustrate the importance of both FUS crosslinking and the inclusion of fibers to the stability of these materials, the stability of granular hydrogels against erosion in vitro of microgel components into surrounding media was compared in three different groups: a FUS-crosslinked granular hydrogel containing microgels and fibers, a FUS-crosslinked granular hydrogel containing only microgels (no fibers), and a granular hydrogel containing microgels and fibers that was not FUS-crosslinked. The crosion was observed by the scaffold breaking apart without fibers compared to obtaining a stable scaffold with fibers. Each resulting hydrogel was incubated on a dish on a rocker for 12 hours and imaged before and after (FIG. 3C). The FUS-crosslinked system containing fibers remained largely intact after 12 hours, whereas the control without fibers exhibited significant erosion. The hydrogels containing fibers and particles that were not exposed to FUS were also not intact after the 12 hour incubation. Based on these observations, the fibers were not only necessary just for stabilizing scaffold porosity in the granular system, but also for allowing FUS sonication to crosslink the particle-based system studied herc.

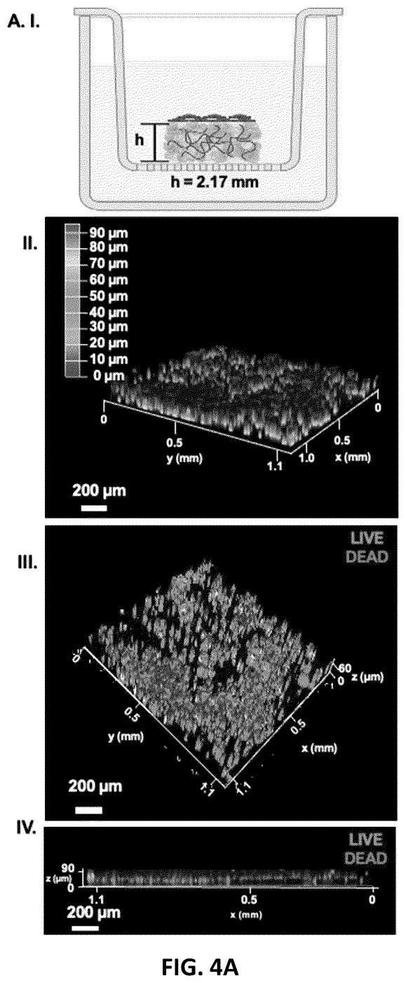

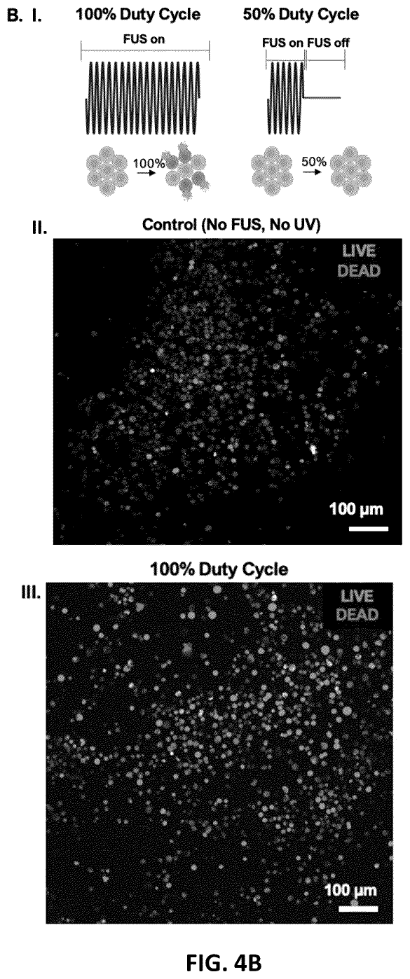

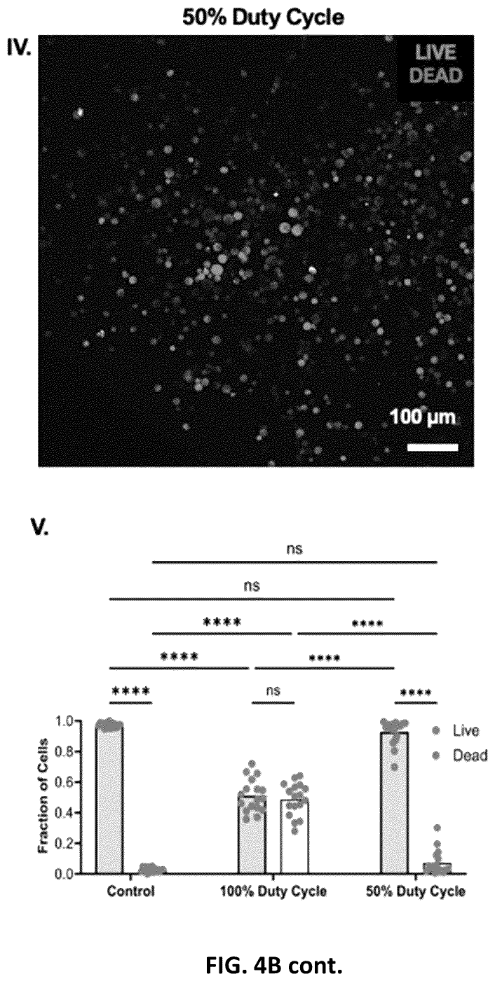

Toward supporting tissue regeneration, the present study assessed in vitro whether cells move into the pore spaces of a FUS crosslinked granular hydrogel using the same ratio of gelatin (75:25 PEG:gelatin microgels) as used in non-cellular FUS experiments. HUVECs were seeded on top of a FUS-crosslinked granular hydrogel (FIG. 4A, I) and observed cellular viability in the scaffold after 48 hours. The majority of HUVECs were in the bottom ˜90 μm of the 2.17 mm-thick scaffold (denoted by red color in depth graph) (FIG. 4A, II), indicating they were able to traverse the highly porous system. Viability staining for living and dead cells indicated that a majority of the HUVECs survived (FIG. 4A, III) and were evenly distributed throughout the bottom of the granular hydrogel scaffold (FIG. 4A, IV). To confirm cells were moving through the scaffold and not flowing around and infiltrating from the bottom, cells in a small volume of medium were applied to the top of the scaffold and observed cell distribution 15 minutes after seeding. Cells were found at the bottom of the scaffold after only 15 minutes, suggesting that large pores sizes were large enough to support convection flow of cell suspended in medium through the material. This degree of porosity should ultimately be supportive of cell migration into scaffolds and tissue growth within the pore spaces when considering in vivo applications. The porosity achieved in this scaffold via gelatin microgels show potential promise for tissue and microvasculature ingrowth in vivo similar to granular hydrogel in vivo work already conducted^{15,31,42,43}

Additionally, the present study assessed HUVEC viability when incorporating within FUS-crosslinked granular hydrogels. Materials underwent FUS at 100% duty cycle for a continuous waveform, where FUS is on for the entire duration of experiment (FIG. 4B, I) and at 50% duty cycle where FUS is on for 0.5 ms and off for 0.5 ms (FIG. 4B, II). It was hypothesized that the 100% duty cycle would lead to cell death and high cellular viability would be maintained at the 50% duty cycle post-FUS. To confirm this, HUVECs were mixed with the granular material compositions at a control condition where the material was exposed to heat alone (37° C. to melt gelatin), 100% FUS duty cycle, and 50% FUS duty cycle. Cell viability was examined via confocal imaging after FUS sonications in the control (FIG. 4B, II), 100% duty cycle (FIG. 4B, III), and 50% duty cycle (FIG. 4B, IV) conditions, where live cells are denoted green and dead cells denoted by magenta colors. When quantified, a 99.5% cell survival was observed in the control condition, whereas ˜50% cells survived at 100% duty cycle and 95% cells survived at 50% duty cycle (FIG. 4B, V). These results together indicate the potential for the present approach to enable minimally invasive tissue regeneration through in situ crosslinking of cell-laden precursor material directly into the desired location.

Drug Release from Granular Hydrogels Using FUS Sonications

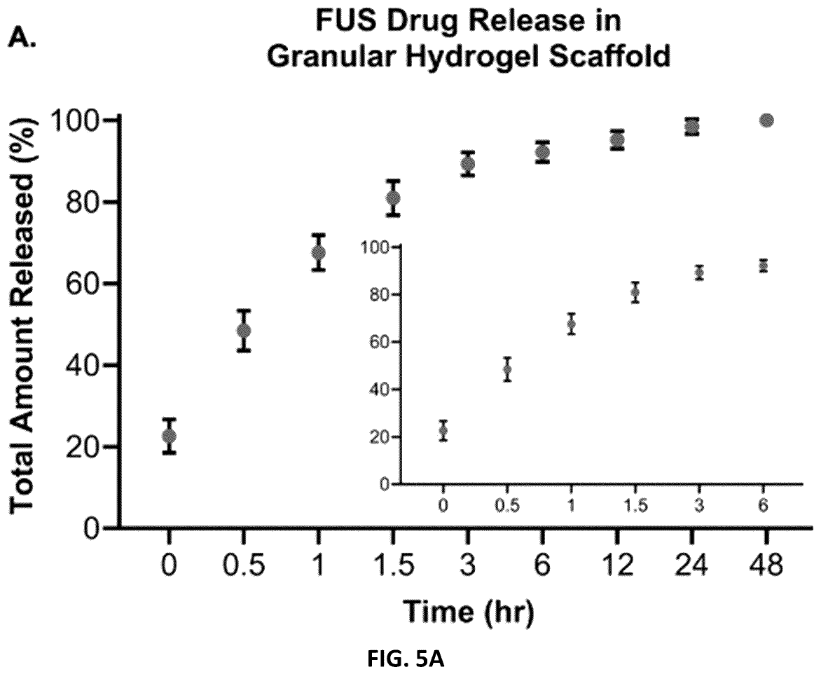

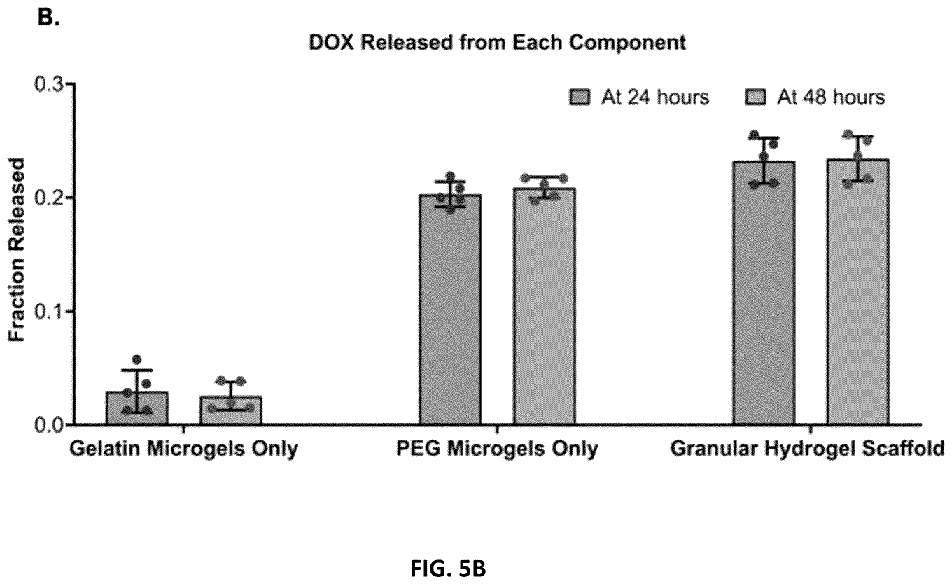

This system has the potential to be used in minimally invasive therapeutic treatments in regenerative medicine applications through the combination with FUS. To enhance its therapeutic effects, the present study explored possibilities for drug release from the granular hydrogel system to support disease applications or regenerative medicine applications where efficacy might be improved by delivering soluble compounds in combination with FUS⁴⁴. A model drug (doxorubicin, DOX) was incorporated into both the PEG and gelatin microgel components separately upon production. FUS could thus be used to induce rapid release from the gelatin particles through melting, with continued release coming from the PEG microgel component within the stabilized system. When quantifying DOX release, it was observed that ˜20% of DOX was released from the granular hydrogel scaffold (FIG. 5A) immediately after FUS sonications within the first 6 hours (FIG. 5A, inset). In samples containing gelatin microgels only (FIG. 5B), melting and 100% release after FUS exposure were observed. While some burst release from the PEG microgels contributes to the burst seen for the combined granular hydrogel system, release from the PEG microgel component continues for days afterwards (FIG. 5A). From the entire granular hydrogel system, it was observed that the gelatin microgels alone released ˜2.5% of the theoretical DOX encapsulated, while the PEG microgels released ˜16% of the theoretical DOX encapsulated after 48 hours (FIG. 5B). The remainder of the theoretical DOX encapsulation in the gelatin microgels was lost in washing steps during processing due to the color change of the microgels across steps.

The combined release of gelatin and PEG post FUS sonication after 24 hours (0.22 fraction release) is less than the released observed over 48 hours (0.23 fraction release) (FIG. 5B), indicating that FUS causes a slow release of DOX from the PEG microgels. This platform should offer high tunability for controlled release regimes through drug encapsulation in FUS-sensitive microgels (here, gelatin microgels), FUS-insensitive microgels (here, PEG microgels), and the possibility to include drugs in the fiber components (not explored here) or in additional microgel components, which can be designed to have different release profiles to create designed drug-release regimes⁴⁵.

### FUS Crosslinking of Granular Hydrogel in Situ

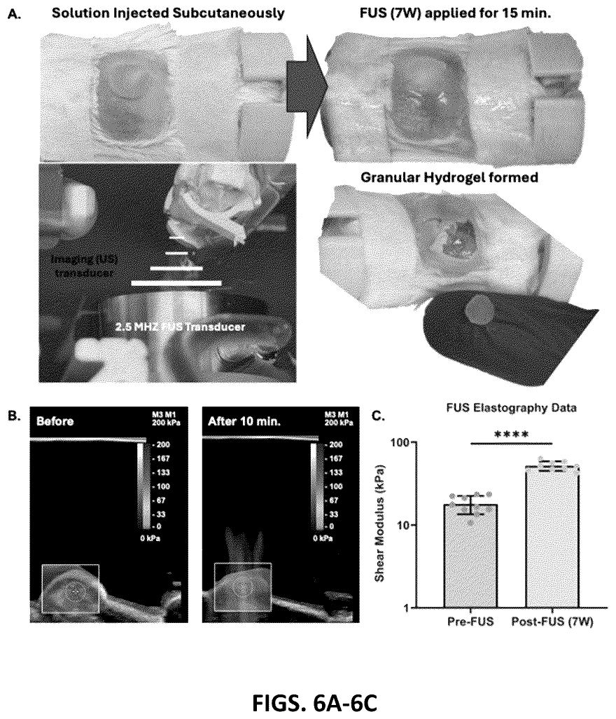

To demonstrate the potential for FUS-induced stabilization of granular hydrogels in vivo, the present study investigated FUS-induced crosslinking of granular hydrogels injected subcutaneously into mouse cadavers. Using a custom ultrasound guided (clastography imaging) FUS system, the present study targeted FUS energy deposition to the center of subcutaneously injected granular hydrogel and then harvested the implant for gross analysis. The present study successfully obtained a crosslinked granular hydrogel formed subcutaneously from a minimally invasive injection followed by FUS sonication. Targeted FUS sonication for 15 minutes yielded a robust material which could be explanted and handled (FIG. 6A). From shear wave elastography imaging (Philips Epiq), the present study was able to guide delivery of FUS energy and obtain an estimate of modulus before FUS sonication (0-33 kPa), during FUS sonication at ˜10 minutes (FIG. 6B) (33-66 kPa), and post sonication for comparison to pre-crosslinking values following established methods⁴⁶. Images from elastography (FIG. 6B) also allowed real time detection of the focal point of the FUS system and demonstrate the potential for guiding FUS application in larger samples or in vivo. Elastography measurements indicated that FUS increased granular hydrogel elastic modulus from ˜30 kPa to ˜80 kPa (FIG. 6C). These in situ stiffness values agree with elastic moduli measured directly in vitro. These results confirm that FUS-induced crosslinking, here observed through increases in bulk mechanics of granular hydrogel, can be achieved in situ in an implant setting. Importantly, clastography imaging of the FUS application also provided insight into the crosslinking process through mechanical readouts, providing potential for monitoring and adjusting the FUS process in real time to further reduce the potential for off-target damage.

In conclusion, an injectable, porous granular hydrogel biomaterial was established that can be delivered and stabilized in situ with non-invasive FUS. Formulation with meltable gelatin microgels allows porosity to be defined in the final scaffold and the fiber component is crucial to stabilizing and maintaining porosity. Parameters for FUS application were defined to leverage the FUS-responsive chemistry in the granular formulation and identified a duty cycle that achieved gelation without compromising cell viability. The resulting material contains a network of mesoscale pores among microgel and fiber components crosslinked to one another at surfaces. Fibrous components were critical to crosslinking, as their absence resulted in rapid deterioration of the gels. These results demonstrate that FUS-induced hydrogels exhibit viscoelastic properties comparable to those produced using traditional photo-initiated crosslinking methods, but that is better poised for initiating in vivo, deep tissue gelation.

### Experiments

Granular Hydrogel Scaffold Fabrication: To create granular hydrogel scaffolds, both PEG microgels and gelatin microgels were first suspended in PBS at 1:1 volume ratio. PEG fibers were suspended in PBS at 1:9 volume ratio. The 300 μL PEG microgels, 100 μL gelatin microgels, and 5% (v/v) PEG fibers were combined. 1.5% (w/v) potassium persulfate (KPS, Sigma), 10% (w/v) PEG-thiol, and 2.27% (w/v) PDEGMA were added to the mixture to achieve a final concentration of 0.2%, 0.7%, and 0.16% respectively. This mixture was then centrifuged at 21,130 rcf for 5 min and excess solution was aspirated off. To crosslink via focused ultrasound, the material was placed in a water bath under FUS sonication for 15 min. To photocrosslink, 10 mM lithium phenyl-2,4,6-trimethylbenzoylphosphinate (LAP) was added in place of KPS to reach a final concentration of 0.7 mM, then crosslinked under 290-320 nm light for 2 min at 10 mW/cm².

Confocal Microscopy: PEG microgels were tagged with thiolated rhodamine-B and PEG fibers were tagged with thiolated 5(6)-carboxyfluorescein amidite (FAM) for visualization. The scaffold crosslinked with UV light was covered in PBS and cultured in a 37° C., humidified incubator for 20 min to liquefy the gelatin microgels prior to imaging. The scaffolds were cut in half then placed in 8 mm confocal dishes for imaging on a Leica Stellaris 5 confocal to assess pore space from melted gelatin. Pore space in the images was quantified via FIJI.

Focused Ultrasound Machine: FUS was applied to microcentrifuge tubes with a single point sonication using a 1.1-MHz FUS transducer (H107, Sonic Concepts, Bethel Washington), driven by an arbitrary function generator (Tektronix, AFG3052C) and amplifier (E&I, 1040L) with either continuous energy or with 10 millisecond burst lengths and varied pulse repetition rates to result in duty cycles ranging from 25-75% for up to 15 min. Both the imaging and treatment transducers were ultrasonically coupled to either the tube containing the precursor solutions or the animal for in situ crosslinking using degassed, deionized water at 37° C. during the duration of each treatment.

Treatment of Mice Cadavers with Focused Ultrasound: Mice cadavers were obtained from other studies, shortly after, they were humanely euthanized with ketamine-xylazine according to approved animal care and use protocols for those planned studies, and prior to planned disposal. The subcutaneous space to which the present injection was targeted was unaffected by the prior studies. The back of each mouse cadaver was shaved with a hair trimmer and hair remover cream. Cadavers were injected with 0.3 mL of granular hydrogel components (PEG microgels, gelatin microgels, PEG fibers, PEG-thiol solution, KPS, and PDEGMA) via subcutaneous injection with an 18G needle. Mouse cadavers were immediately positioned in a custom ultrasound guided FUS system with a 2.5 Mhz transducer (H147, Sonic Concepts). The injected hydrogel precursor was located with the imaging transducer and FUS was then applied to the center of the site with a single point sonication at a 0.6 MPa peak negative pressure for 15 min with continuous waveform.

Rheological Analysis of Granular Hydrogel Properties: Granular hydrogel properties were characterized using rheological studies with a stress-controlled DHR-2 rheometer (TA Instruments), equipped with a 20 mm sandblasted parallel plate. Granular hydrogels were placed onto the bottom plate of the rheometer. Oscillatory tests were performed once the storage modulus reached an equilibrium valuc.

Compressive Analysis of Granular Hydrogel Mechanics: Continuous, bulk hydrogel and granular hydrogel properties were also characterized on an Instron using compression at 0.5 mm/s rate with a 10 N load cell on top. The region of 0.1-0.2 strain of each measurement was taken and the stress-strain curve was plotted to find the best fit line, or compressive moduli for the data.

Statistical Analyses: All results reported with error bars are means with standard deviation. The “n” values per group are made by individual data points shown. Statistical significance was assessed at p<0.05 for all experiments and were calculated using GraphPad Prism. Statistical tests are provided in the figure legends.

PEG Microgels and Electrospinning Parameters: Experimental procedures for PEG microgels and electrospun scaffold formation are provided below.

Synthesis of poly(di(ethylene glycol) methyl ether methacrylate) (PDEGMA): Di(ethylene glycol) methyl ether methacrylate (DEGMA, stabilized with monomethyl ether hydroquinone), 4-Cyano-4-(phenylcarbonothioylthio)pentanoic acid (CTA), 4-4′-Azobis(4-cyanovaleric acid) (ACVA, ≥98.0%), and tetrahydrofuran (THF, ≥99.0%, stabilized with butylated hydroxytoluene) were purchased from Sigma-Aldrich. DEGMA was purified by passing the monomer through a column packed with aluminum oxide to remove inhibitor prior to polymerization. All other reagents were used as received. Poly(di(ethylene glycol) methyl ether methacrylate) was synthesized via reversible-addition fragmentation-chain transfer (RAFT) polymerization. DEGMA (2.8 mL, 15 mmol), CTA (55.9 mg, 0.2 mmol), ACVA (5.6 mg, 0.02 mmol) and tetrahydrofuran (8.3 mL, 25 v/v % of monomer concentration) were added synchronously to a 20 mL scintillation vial. The solution was degassed with nitrogen for 30 min, then added to a silicone oil bath set to 70° C. for 4 h. The solution was purified via dialysis against 40/60 methanol/water, then against water. The polymer was then lyophilized and characterized using ¹H nuclear magnetic resonance (NMR) spectroscopy and size exclusion chromatography (SEC) in trifluoroethanol (TFE) containing 0.02 M sodium trifluoroacetate (NaTFAc).

Electrospun PEG Nanofibers Fabrication: PEG-norbornene electrospun hydrogel nanofibers were produced and adapted from previous literature²⁸. Electrospinning hydrogel solution containing 10% (w/v) 8-arm PEG-Norbornene, 5% (w/v) 900 kDa PEO, 4-arm PEG-thiol ([thiol]:[norbornene]=0.6), and 0.05% Irgacure 2959 in deionized water was dissolved overnight. The 0.6 ratio of thiol to norbornene was chosen to allow for excess norbornenes upon further conjugation of peptides to the fiber surface and secondary crosslinking within the fiber-reinforced granular hydrogel scaffold. A co-spin solution containing 4% (w/v) 900 kDa PEO in deionized water was dissolved overnight. The hydrogel solution was extruded using a syringe pump at a flow rate of 0.5 ml/h through a 16G needle to separate individual PEG fibers. The co-spin solution was also extruded using a syringe pump at a flow rate of 0.5 mL/h through a 16G needle. The second syringe pump was positioned on the opposite side of the mandrel from the hydrogel solution to minimize potentially disruptive interactions between fibers produced from the two different solutions during processing. The fibers were collected on an aluminum foil substrate attached to a mandrel spinning at ˜1,000 rpm. 12 kV positive voltage was applied to the hydrogel needle, 6 kV positive voltage was applied to the co-spin needle, and 4 kV negative voltage was applied to the collection substrate.

After collection, the collected fibers on the aluminum foil were crosslinked under UV light for 15 min at 5 mW/cm² under N_{2(g)} atmosphere. Once crosslinked, the fibers were hydrated with deionized water to detach them from the collection substrate. The resulting material was then suspended in IX PBS. The suspension was homogenized at 10,000 rpm for 2 min to segment fibers and then filtered through a 40 μm cell strainer (Fisher) to eliminate aggregations of fibers.

PEG Microgel Fabrication: Microgels were produced as previously described⁴⁷. Precursor solution containing 6% (w/v) PEG-acrylate (4-arm, 10 kDa, JenKem) and 4% (w/v) PEG-thiol (4-arm, 10 kDa, JenKem) in 1X Dulbecco's phosphate buffered saline (DPBS) was vortexed to dissolve. The solution was placed on a syringe pump at a flow rate of 40 μL/min through a 30G needle into a spinning oil/base solution. Oil/base solution containing light mineral oil, 1% (v/v) Span80, and 0.5% (v/v) triethanolamine (TEOA) in light mineral oil was spun on a homogenizer at 2,000 rpm. The microgels were left spinning for 1 hour post solution dispensed. Microgels were then washed three times with isopropanol via centrifugation at 3,200 rcf for 5 min each, then hydrated overnight in 1X DPBS to fully swell. The suspension of microgels were centrifuged at 3,200 rcf for 5 min and rehydrated again in 1X DPBS before aliquoting into a 1:1 microgel:1X DPBS ratio.

Gelatin Microgel Fabrication: Gelatin from bovine skin was dissolved in 1X PBS solution at 15% (w/v) at ˜80° C. The solution was then added to light mineral oil with 2% (v/v) Span 80 heated to ˜80° C. and emulsified at 2,000 rpm for 3 min. The emulsion was then allowed to cool to room temperature to form gelatin microgels. Once cooled, the gelatin microgels were first centrifuged at 3,000 ref for 5 min. After discarding the supernatant oil, the microgels were suspended in 2-3 mL of 2% (v/v) Pluronic F-127 in 1X PBS to the pellet and centrifuged at 3,200 rcf for 2 min. Then, the microgels were washed 8× in 1X PBS and centrifuged at 3,000 rcf for 2 min after each wash. Finally, the gelatin microgels were filtered through a cell strainer (pluriStrainer, Pluriselect) with mesh sizes ˜1.5X the largest diameter microgel in the distribution (40 μm) to eliminate clusters formed through aggregation.

Scanning Electron Microscopy (SEM): The granular hydrogels were flashed frozen with liquid nitrogen then lyophilized overnight. The lyophilized granular hydrogel was placed on a carbon tab in 0.5 inches (12 mm) (Al) SEM stubs and imaged on the Quanta 650 (FEI) with a field emission gun source operating in low vacuum mode.

Drug Encapsulation in Granular Hydrogel: Doxorubicin (DOX) was encapsulated into gelatin and PEG precursor solutions separately to reach a final concentration of 1 mg/mL. To incorporate DOX into the gelatin microgels, gelatin from bovine skin was dissolved in 1 mg/mL DOX solution at 15% (w/v) at ˜80° C. in DI water. Gelatin microgels were washed and processed as described above. To incorporate DOX into the PEG microgels, 12% (w/v) 4-arm PEG-acrylate and 8% (w/v) 4-arm PEG-thiol solutions were mixed with 2 mg/mL DOX solution to obtain final concentrations of 6% PEG-acrylate, 4% PEG-thiol, and 1 mg/mL DOX. The PEG microgels were crosslinked and washed. PEG microgels, PEG fibers, and gelatin microgels were placed in microcentrifuge tubes at the desired volume ratios and mixed with PDEGMA, KPS, and PEG-thiol solution as described above. The granular hydrogel material underwent FUS sonications for 10 min. Immediately, 1X PBS was placed on the samples to obtain a tO time point for DOX released. Controlled volumes of PBS were pipetted out of the tube at various timepoints over 48 h.

Quantification of Drug Release: Samples taken from each timepoint of the drug release experiment were saved in microcentrifuge tubes and stored in the refrigerator (4° C.) until quantification. All samples were pipetted into a 96 wellplate for absorbance measurements. These measurements were conducted on plate reader (Multimode Microplate, Tecan Spark) at 475 nm light to calculate the absorbance values of DOX within each sample. A standard curve was generated by preparing DOX solutions of known concentrations as low as 0.006 μg/mL. A best fit curve was obtained from plotting absorbance values of the known concentrations in GraphPad Prism with an r-squared value of 0.998. The best fit equation (multivariable analysis was used in DOX release experiments to obtain an amount of material in solution at each time point. Further calculation was conducted to get the percent fraction of DOX released over time within the system.

Continuous Hydrogel Fabrication: A continuous, bulk hydrogel was created using a 20% (v/v) PEG-diacrylate (PEGDA) solution in DI water (700 Da, Sigma), 2.27% (w/v) PDEGMA, and 1.5% (w/v) KPS solutions. This solution was sonicated with FUS until a hydrogel formed, which resulting in an opaque color of solution. To photocrosslink the continuous hydrogel, 10 mM LAP was added in place of KPS as described above, then crosslinked for 2 min under 290-320 nm at 10 mW/cm².

Image Analysis and Porosity Quantification: Images were taken using a Leica DMi8 widefield microscope using a 5X objective and Leica Stellaris 5 confocal microscope using a 10X objective. Images taken on the confocal were stained with calcein-AM for live cells and cthidium homoimer-1 for dead cells. Porosity images were processed using FIJI software.

Cell Staining: In experiments measuring cell viability, cells were stained using a LIVE/DEAD assay kit (ThermoFisher, L3224) that used calcein-AM and ethidium homoimer-1 to stain live and dead cells, respectively.

### References for Example 1

(1) Chen, Z.; Wang, L.; Guo, C.; Qiu, M.; Cheng, L.; Chen, K.; Qi, J.; Deng, L.; He, C.; Li, X.; Yan, Y. Vascularized Polypeptide Hydrogel Modulates Macrophage Polarization for Wound Healing. Acta Biomater 2023, 155, 218-234. https://doi.org/10.1016/J.ACTBIO.2022.11.002.
(2) Dimatteo, R.; Darling, N. J.; Segura, T. In Situ Forming Injectable Hydrogels for Drug Delivery and Wound Repair. Adv Drug Deliv Rev 2018, 127, 167-184. https://doi.org/10.1016/J.ADDR.2018.03.007.
(3) Li, J.; Chen, G.; Xu, X.; Abdou, P.; Jiang, Q.; Shi, D.; Gu, Z. Advances of Injectable Hydrogel-Based Scaffolds for Cartilage Regeneration. Regen Biomater 2019, 6(3), 129-140. https://doi.org/10.1093/rb/rbz022.
(4) Wei, W.; Ma, Y.; Yao, X.; Zhou, W.; Wang, X.; Li, C.; Lin, J.; He, Q.; Leptihn, S.; Ouyang, H. Advanced Hydrogels for the Repair of Cartilage Defects and Regeneration. Bioact Mater 2021, 6(4), 998-1011. https://doi.org/10.1016/J.BIOACTMAT.2020.09.030.
(5) Hasan, A.; Khattab, A.; Islam, M. A.; Hweij, K. A.; Zeitouny, J.; Waters, R.; Sayegh, M.; Hossain, M. M.; Paul, A. Injectable Hydrogels for Cardiac Tissue Repair after Myocardial Infarction. Advanced Science 2015, 2(11), 1500122. https://doi.org/10.1002/ADVS.201500122.
(6) Madhusudanan, P.; Raju, G.; Shankarappa, S. Hydrogel Systems and Their Role in Neural Tissue Engineering. J R Soc Interface 2020, 17(162). https://doi.org/10.1098/RSIF.2019.0505.
(7) Constantinou, A. P.; Sam-Soon, N. F.; Carroll, D. R.; Georgiou, T. K. Thermoresponsive Tetrablock Terpolymers: Effect of Architecture and Composition on Gelling Behavior. Macromolecules 2018, 51(18), 7019-7031. https://doi.org/10.1021/ACS.MACROMOL.8B01251/ASSET/IMAGES/LARGE/MA-2018-01251M_0010.JPEG.
(8) Sung, J.; Lee, D. G.; Lee, S.; Park, J.; Jung, H. W. Crosslinking Dynamics and Gelation Characteristics of Photo- and Thermally Polymerized Poly (Ethylene Glycol) Hydrogels. Materials 2020, 13(15). https://doi.org/10.3390/MA13153277.
(9) Wu, W. X.; Huang, Y. C.; Lec, W. F. Effect of Poly(Ethylene Glycol)-Derived Crosslinkers on the Properties of Thermosensitive Hydrogels. Iranian Polymer Journal (English Edition) 2020, 29(8), 679-691. https://doi.org/10.1007/s13726-020-00831-7.
(10) Lin, F.; Yu, J.; Tang, W.; Zheng, J.; Defante, A.; Guo, K.; Wesdemiotis, C.; Becker, M. L. Peptide-Functionalized Oxime Hydrogels with Tunable Mechanical Properties and Gelation Behavior. Biomacromolecules 2013, 14(10), 3749-3758. https://doi.org/10.1021/BM401133R/SUPPL_FILE/BM401133R_SI_001.PDF.
(11) Ash, C.; Dubec, M.; Donne, K.; Bashford, T. Effect of Wavelength and Beam Width on Penetration in Light-Tissue Interaction Using Computational Methods. Lasers Med Sci 2017, 32 (8), 1909-1918. https://doi.org/10.1007/S10103-017-2317-4.
(12) Mironi-Harpaz, I.; Wang, D. Y.; Venkatraman, S.; Seliktar, D. Photopolymerization of Cell-Encapsulating Hydrogels: Crosslinking Efficiency versus Cytotoxicity. Acta Biomater 2012, 8(5), 1838-1848. https://doi.org/10.1016/J.ACTBIO.2011.12.034.
(13) Cai, B.; Gu, H.; Wang, F.; Printon, K.; Gu, Z.; Hu, X. Ultrasound Regulated Flexible Protein Materials: Fabrication, Structure and Physical-Biological Properties. Ultrason Sonochem 2021, 79, 105800. https://doi.org/10.1016/J.ULTSONCH.2021.105800.
(14) Sheybani, N. D.; Price, R. J. Perspectives on Recent Progress in Focused Ultrasound Immunotherapy. Theranostics 2019, 9(25), 7749-7758. https://doi.org/10.7150/thno.37131.
(15) Griffin, D. R.; Weaver, W. M.; Scumpia, P. O.; Di Carlo, D.; Segura, T. Accelerated Wound Healing by Injectable Microporous Gel Scaffolds Assembled from Annealed Building Blocks. Nat Mater 2015, 14(7), 737-744. https://doi.org/10.1038/nmat4294.
(16) Nele, V.; Schutt, C. E.; Wojciechowski, J. P.; Kit-Anan, W.; Doutch, J. J.; K Armstrong, J. P.; Stevens, M. M.; Nele, V.; Schutt, C. E.; Wojciechowski, J. P.; Kit-Anan, W.; K Armstrong, J. P.; Stevens, M. M.; Doutch, J. J. Ultrasound-Triggered Enzymatic Gelation. Advanced Materials 2020, 32(7), 1905914. https://doi.org/10.1002/ADMA.201905914.
(17) Yang, S. R.; Ych, Y. Y.; Ych, Y. C. Ultrasound-Triggered Hydrogel Formation through Thiol-Norbornene Reactions. Chemical Communications 2022, 58(8), 1119-1122. https://doi.org/10.1039/D1CC04848A.
(18) Arrizabalaga, J. H.; Smallcomb, M.; Abu-Laban, M.; Liu, Y.; Yeingst, T. J.; Dhawan, A.; Simon, J. C.; Hayes, D. J. Ultrasound-Responsive Hydrogels for On-Demand Protein Release. ACS Appl Bio Mater 2022, 5(7), 3212-3218. https://doi.org/10.1021/ACSABM.2C00192/SUPPL_FILE/MT2C00192_SI_001.PDF.
(19) Rokita, B.; Rosiak, J. M.; Ulanski, P. Ultrasound-Induced Cross-Linking and Formation of Macroscopic Covalent Hydrogels in Aqueous Polymer and Monomer Solutions. https://doi.org/10.1021/ma802565p.
(20) Dalecki, D.; Hocking, D. C. Ultrasound Technologies for Biomaterials Fabrication and Imaging. Annals of Biomedical Engineering 2014 43:3 2014, 43(3), 747-761. https://doi.org/10.1007/S10439-014-1158-6.
(21) Rice, M. A.; Waters, K. R.; Anseth, K. S. Ultrasound Monitoring of Cartilaginous Matrix Evolution in Degradable PEG Hydrogels. Acta Biomater 2009, 5(1), 152. https://doi.org/10.1016/J.ACTBIO.2008.07.036.
(22) Garvin, K. A.; Vanderburgh, J.; Hocking, D. C.; Dalecki, D. Controlling Collagen Fiber Microstructure in Three-Dimensional Hydrogels Using Ultrasound. https://doi.org/10.1121/1.4812868.
(23) Lee, J. B.; Jeong, S. I.; Bae, M. S.; Yang, D. H.; Heo, D. N.; Kim, C. H.; Alsberg, E.; Kwon, I. K. Highly Porous Electrospun Nanofibers Enhanced by Ultrasonication for Improved Cellular Infiltration. Tissue Eng Part A 2011, 17(21-22), 2695-2702. https://doi.org/10.1089/TEN.TEA.2010.0709.
(24) Gao, Z.; Zhu, H.; Li, X.; Zhang, P.; Ashokkumar, M.; Cavalieri, F.; Hao, J.; Cui, J. Sono-Polymerization of Poly(Ethylene Glycol)-Based Nanoparticles for Targeted Drug Delivery. ACS Macro Lett 2019, 8(10), 1285-1290. https://doi.org/10.1021/ACSMACROLETT.9B00576/ASSET/IMAGES/LARGE/MZ9B00576_0 003.JPEG.
(25) Kuang, X.; Rong, Q.; Belal, S.; Vu, T.; López López, A. M.; Wang, N.; Arcan, M. O.; Garciamendez-Mijares, C. E.; Chen, M.; Yao, J.; Zhang, Y. S. Self-Enhancing Sono-Inks Enable Deep-Penetration Acoustic Volumetric Printing. Science (1979) 2023, 382 (6675), 1148-1155. https://doi.org/10.1126/science.adi1563.
(26) Gorick, C. M.; Chappell, J. C.; Price, R. J. Applications of Ultrasound to Stimulate Therapeutic Revascularization. Int J Mol Sci 2019, 20(12). https://doi.org/10.3390/IJMS20123081.
(27) Gorick, C. M.; Breza, V. R.; Nowak, K. M.; Cheng, V. W. T.; Fisher, D. G.; Debski, A. C.; Hoch, M. R.; Demir, Z. E. F.; Tran, N. M.; Schwartz, M. R.; Sheybani, N. D.; Price, R. J. Applications of Focused Ultrasound-Mediated Blood-Brain Barrier Opening. Adv Drug Deliv Rev 2022, 191, 114583. https://doi.org/10.1016/J.ADDR.2022.114583.
(28) Sun, Y.; Chen, L. G.; Fan, X. M.; Pang, J. L. Ultrasound Responsive Smart Implantable Hydrogels for Targeted Delivery of Drugs: Reviewing Current Practices. Int J Nanomedicine 2022, 17, 5001. https://doi.org/10.2147/IJN.S374247.
(29) Dilley, R.; Yeingst, C.: ; Helton, T. J.; Rawnaque, A. M.; Arrizabalaga, F. S.; Ravnic, J. H.; Simon, D. J.; Hayes, J. C.; Yeingst, T. J.; Helton, A. M.; Rawnaque, F. S.; Arrizabalaga, J. H.; Ravnic, D. J.; Simon, J. C.; Hayes, D. J. Focused Ultrasound-Mediated Release of Bone Morphogenetic Protein 2 from Hydrogels for Bone Regeneration. Gels 2025, Vol. 11, Page 120 2025, 11(2), 120. https://doi.org/10.3390/GELS11020120.
(30) Wang, Y.; Kohane, D. S. External Triggering and Triggered Targeting Strategies for Drug Delivery. Nature Reviews Materials 2017 2:6 2017, 2(6), 1-14. https://doi.org/10.1038/natrevmats.2017.20.
(31) Griffin, D. R.; Archang, M. M.; Kuan, C. H.; Weaver, W. M.; Weinstein, J. S.; Feng, A. C.; Ruccia, A.; Sideris, E.; Ragkousis, V.; Koh, J.; Plikus, M. V.; Di Carlo, D.; Segura, T.; Scumpia, P. O. Activating an Adaptive Immune Response from a Hydrogel Scaffold Imparts Regenerative Wound Healing. Nature Materials 2020 20:4 2020, 20(4), 560-569. https://doi.org/10.1038/s41563-020-00844-w.
(32) Whitewolf, J.; Riley, L.; Brady, B.; Grewal, G.; Segura, T.; Highley, C. Highly Porous Granular Hydrogels Reinforced by Electrospun Hydrogel Fibers for Long-Term Stability. bioRxiv 2024, 2024.10.28.620615. https://doi.org/10.1101/2024.10.28.620615.
(33) Xin, S.; Deo, K. A.; Dai, J.; Pandian, N. K. R.; Chimene, D.; Moebius, R. M.; Jain, A.; Han, A.; Gaharwar, A. K.; Alge, D. L. Generalizing Hydrogel Microparticles into a New Class of Bioinks for Extrusion Bioprinting. Sci Adv 2021, 7(42), 3087-3102. https://doi.org/10.1126/sciadv.abk3087.
(34) Habibi, M.; Foroughi, S.; Karamzadeh, V.; Packirisamy, M. Direct Sound Printing. Nature Communications 2022 13:1 2022, 13(1), 1-11. https://doi.org/10.1038/s41467-022- 29395-1.
(35) Garvin, K. A.; Dalecki, D.; Hocking, D. C. Vascularization of Three-Dimensional Collagen Hydrogels Using Ultrasound Standing Wave Fields. Ultrasound Med Biol 2011, 37 (11), 1853. https://doi.org/10.1016/J.ULTRASMEDBIO.2011.07.008.
(36) Ashokkumar, M. The Characterization of Acoustic Cavitation Bubbles—An Overview. Ultrason Sonochem 2011, 18(4), 864-872. https://doi.org/10.1016/J.ULTSONCH.2010.11.016.
(37) Kim, G.; Wu, Q.; Chu, J. L.; Smith, E. J.; Oelze, M. L.; Moore, J. S.; Li, K. C. Ultrasound Controlled Mechanophore Activation in Hydrogels for Cancer Therapy. Proc Natl Acad Sci U S A 2022, 119(4), e2109791119. https://doi.org/10.1073/PNAS.2109791119/SUPPL_FILE/PNAS.2109791119.SM01.AVI.
(38) Yamaguchi, S.; Higashi, K.; Azuma, T.; Okamoto, A. Supramolecular Polymeric Hydrogels for Ultrasound-Guided Protein Release. Biotechnol J 2019, 14(5), 1800530. https://doi.org/10.1002/BIOT.201800530.
(39) Luo, J.; Fang, Z.; Smith, R. L.; Qi, X.; Luo, J.; Fang,. Z; Smith, R. L.; Qi, X. Fundamentals of Acoustic Cavitation in Sonochemistry. Biofuels and Biorefineries 2015, 4, 3-33. https://doi.org/10.1007/978-94-017-9624-8_1.
(40) Lyon, P. C.; Mannaris, C.; Gray, M.; Carlisle, R.; Gleeson, F. V.; Cranston, D.; Wu, F.; Coussios, C. C. Large-Volume Hyperthermia for Safe and Cost-Effective Targeted Drug Delivery Using a Clinical Ultrasound-Guided Focused Ultrasound Device. Ultrasound Med Biol 2021, 47(4), 982-997. https://doi.org/10.1016/J.ULTRASMEDBIO.2020.12.008.
(41) Watson, N. J.; Johal, R. K.; Reinwald, Y.; White, L. J.; Ghacmmaghami, A. M.; Morgan, S. P.; Rose, F. R. A. J.; Povey, M. J. W.; Parker, N. G. Post-Processing of Polymer Foam Tissue Scaffolds with High Power Ultrasound: A Route to Increased Pore Interconnectivity, Pore Size and Fluid Transport. Materials Science and Engineering C 2013, 33(8), 4825-4832. https://doi.org/10.1016/j.msec.2013.07.046.
(42) Pructt, L. J.; Jenkins, C. H.; Singh, N. S.; Catallo, K. J.; Griffin, D. R. Heparin Microislands in Microporous Annealed Particle Scaffolds for Accelerated Diabetic Wound Healing. Adv Funct Mater 2021, 31(35). https://doi.org/10.1002/ADFM.202104337.
(43) Koh, J.; Griffin, D. R.; Archang, M. M.; Feng, A. C.; Horn, T.; Margolis, M.; Zalazar, D.; Segura, T.; Scumpia, P. O.; Di Carlo, D. Enhanced In Vivo Delivery of Stem Cells Using Microporous Annealed Particle Scaffolds. Small 2019, 15(39). https://doi.org/10.1002/smll.201903147.
(44) Khan, Z. M.; Zhang, J.; Gannon, J.; Johnson, B. N.; Verbridge, S. S.; Vlaisavljevich, E. Development of an Injectable Hydrogel for Histotripsy Ablation Toward Future Glioblastoma Therapy Applications. Ann Biomed Eng 2024, 52(12), 3157-3171. https://doi.org/10.1007/S10439-024-03601-1/FIGURES/9.
(45) Mealy, J. E.; Rodell, C. B.; Burdick, J. A. Sustained Small Molecule Delivery from Injectable Hyaluronic Acid Hydrogels through Host-Guest Mediated Retention. J Mater Chem B 2015, 3(40), 8010-8019. https://doi.org/10.1039/C5TB00981B.
(46) Sultan, S. R.; Alghamdi, A.; Abdeen, R.; Almutairi, F. Evaluation of Ultrasound Point Shear Wave Elastography Reliability in an Elasticity Phantom. Ultrasonography 2021, 41(2), 291. https://doi.org/10.14366/USG.21114.
(47) Claxton, N. L.; Luse, M. A.; Isakson, B. E.; Highley, C. B. Engineering Granular Hydrogels without Interparticle Cross-Linking to Support Multicellular Organization. ACS Biom Sci Eng 2024. https://doi.org/10.1021/ACSBIOMATERIALS.4C01563/ASSET/IMAGES/LARGE/AB4C01563_0007.JPEG.

### Example 2

Revascularization of tissues is often required to restore tissue function and heal wounds, for example in cases of ischemic injury. Recent regeneration therapy has shown promise of using porous, hydrogel microparticle (microgel) scaffolds to treat tissue loss and support revascularization¹⁻⁴. Delivering porous scaffolds often requires chemical stabilization of this porosity, which can be challenging to trigger at deep tissue locations. Focused ultrasound (FUS) has emerged as a means for not only directly inducing revascularization, but also used for inducing biomaterials crosslinking⁵⁻⁹. In clinical applications, FUS has been used as a treatment modality in many diseases and is currently used in vivo in numerous applications and studies^{10,11}. Herein, the potential of FUS to stabilize granular hydrogels and simultaneously induce the formation of porosity through combining crosslinking and heating effects on a novel hydrogel system is demonstrated. Whether a composite microgel scaffold consisting of thermally responsive gelatin particles and particles containing FUS-responsive acrylate chemistry can be used to create a crosslinked porous scaffold after FUS-treatment is investigated.

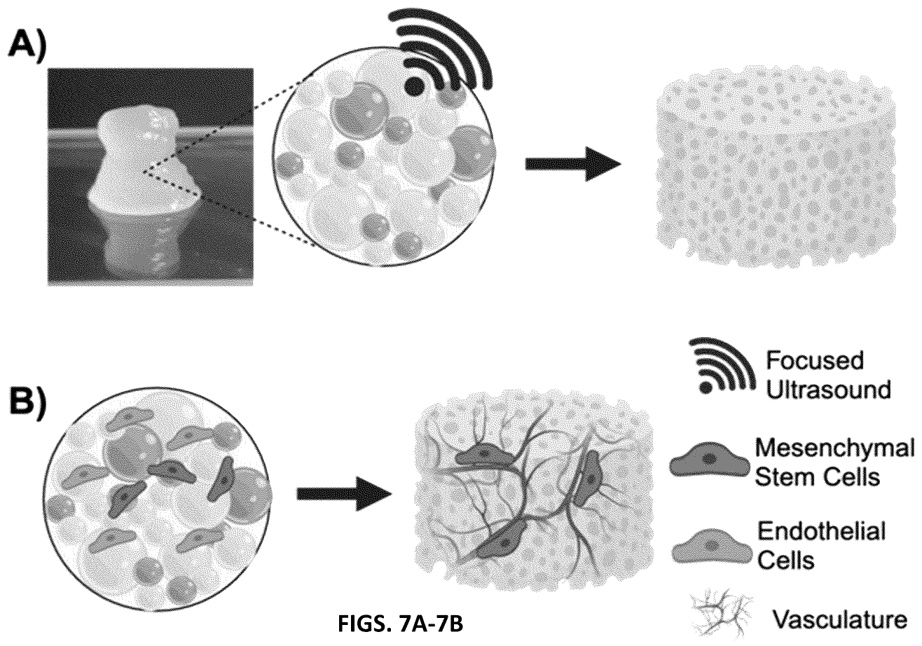

FIG. 7 illustrates the overall concept and functional outcome of the granular hydrogel system. FIG. 7A shows the schematic project workflow, where two distinct subpopulations of microgels, gelatin-based (purple) and PEG-based (yellow), are mixed together.

When this mixture is exposed to FUS, the energy triggers crosslinking between the microgels, resulting in the formation of a cohesive granular hydrogel. Panel B then depicts the functional environment created within this scaffold, where co-cultured cells are supported within the microgel matrix. This environment promotes cell-cell interactions and spatial organization, enabling the emergence of microvascular structures, which highlights the potential of the system for applications in tissue engineering and regenerative medicine.

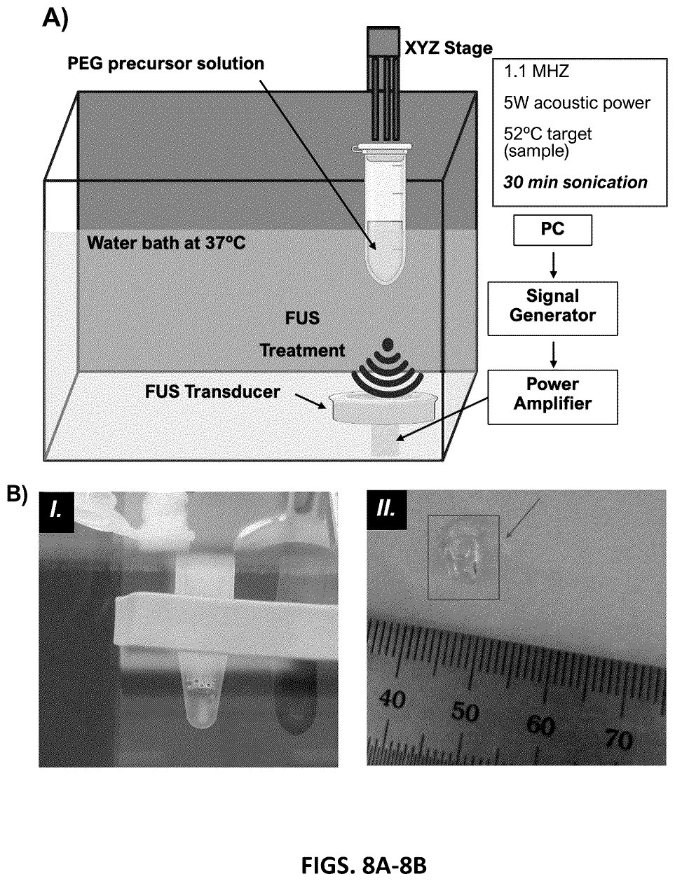

Referring to FIGS. 8A and 8B, a schematic illustrates the focused ultrasound setup, wherein a precursor solution of PEG-acrylate is combined with an initiator and positioned in a heated water bath at the ultrasound's focal point. The solution undergoes sonication at various timepoints using a 1.1 MHz FUS transducer until reaching a target temperature of 52° C. As demonstrated in the images of continuous hydrogels, the gelation of PEG precursor material under FUS sonication is visible, with a white opaque area indicating the onset of crosslinking (FIG. 8A), which subsequently disappears once the gel is removed from the FUS stimuli (FIG. 8B).

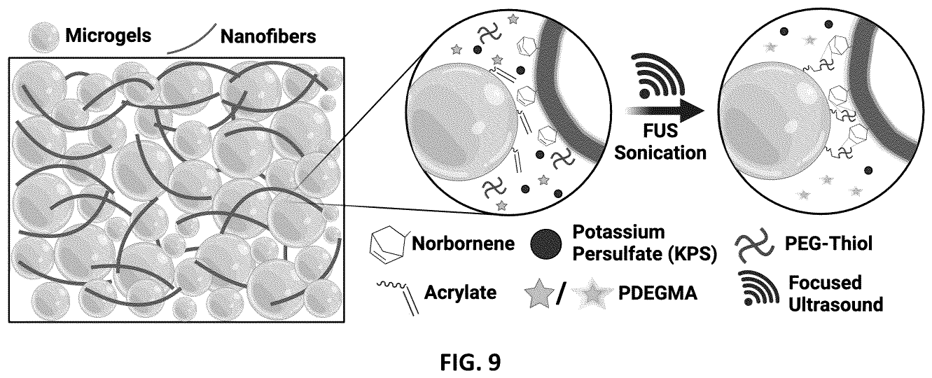

FIG. 9 depicts the focused ultrasound crosslinking mechanism used to create granular hydrogel scaffolds. In this process, PEG microgels, which contain excess acrylates, are combined with PEG nanofibers that include excess norbornenes. The mixture also incorporates a PEG-thiol solution, an initiator (potassium persulfate), and a thermo-responsive material (PDEGMA). These components are then centrifuged at high speed to remove any excess liquid. Following this preparation, focused ultrasound sonication is applied at various time points, resulting in the formation of a crosslinked granular scaffold. The final product closely resembles a continuous, bulk hydrogel, demonstrating the effective crosslinking and integration of the system.

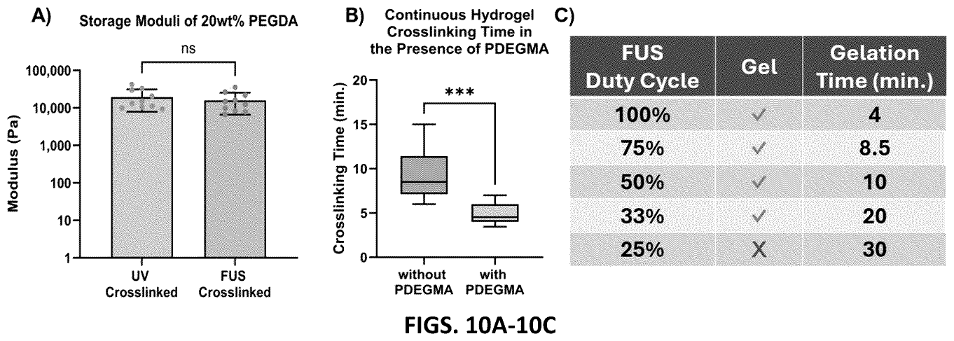

Referring to FIG. 10, rheological measurements demonstrate that both UV and FUS crosslinking of a continuous bulk hydrogel produce comparable mechanical properties (FIG. 10A). When incorporating a thermoresponsive material, PDEGMA, the crosslinking time under FUS stimulation is notably reduced (FIG. 10B), with statistical analysis conducted using GraphPad Prism and an unpaired T-test revealing significant differences (p<0.001). Various FUS duty cycles were investigated, as detailed in FIG. 10C, with results showing that a crosslinked continuous hydrogel can be successfully achieved using a low duty cycle of 33%, wherein FUS is active for 0.33 ms and inactive for 0.67 ms.

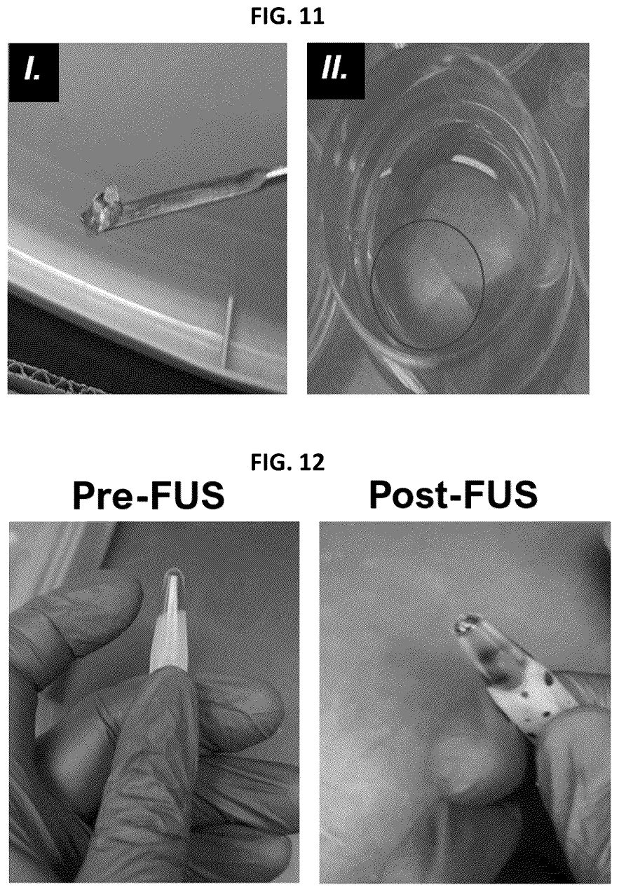

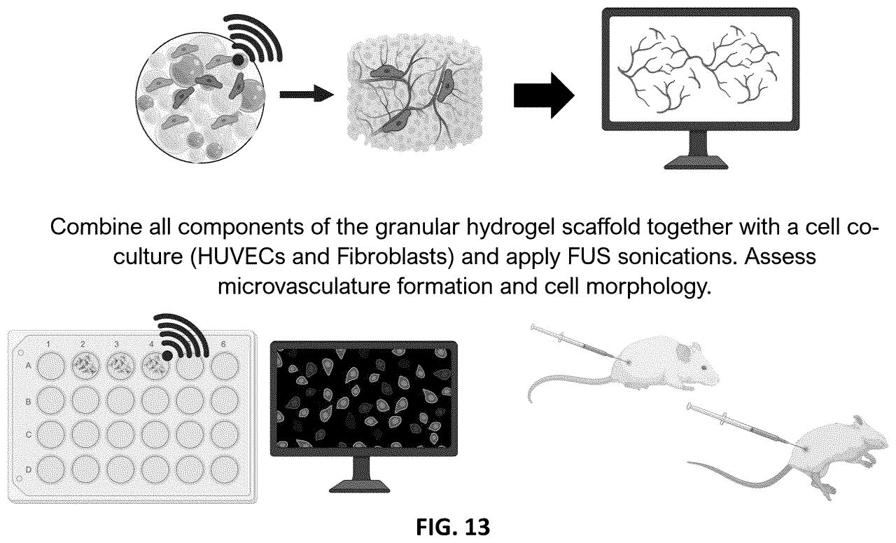

FIG. 11 shows the FUS-induced crosslinked granular scaffold being lifted with a spatula after extraction from an Eppendorf tube (I). The scaffold maintains its structural integrity even when immersed in an excess of 1X PBS solution, as highlighted by the hydrogel enclosed within the red circle (II).

FIG. 12 illustrates gelatin microgels containing India ink (black) before and after FUS stimulation. Initially, the microgels are densely packed within an Eppendorf tube. Following FUS stimulation, which raises the temperature above the target threshold of 37° C., the gelatin microgels undergo a phase transition, liquefying into a homogeneous solution.

### References for Example 2

(1) Griffin, D. R., et. al. Nat Mater 14, 737-744 (2015).
(2) Lupo, G. et al. (2020) doi:10.3389/fcell.2020.579659.
(3) Moon, J. J. et al. (2010) doi:10.1016/j.biomaterials.2010.01.104.
(4) Chapla, R. & West, J. L. Progress in Biomedical Engineering 3, 012002 (2021).
(5) Garvin, K. A., et. al. doi:10.1121/1.4812868.
(6) Dalecki, D. & Hocking, D. C. Annals of Biomedical Engineering 2014 43:3 43, 747-761 (2014).
(7) Yang, S. R., Yeh, Y. Y. & Yeh, Y. C. Chemical Communications 58, 1119-1122 (2022).
(8) Garvin, K. A., Dalecki, D. & Hocking, D. C. Ultrasound Med Biol 37, 1853 (2011).
(9) Kuang, X. et al. Science (1979) 382, 1148-1155 (2023).
(10) Gorick, C. M., Chappell, J. C. & Price, R. J. Int J Mol Sci 20, (2019).
(11) Schwartz, M. R., Debski, A. C. & Price, R. J. Journal of Controlled Release 339, 531-546 (2021).

1. A method of preparing a crosslinked granular hydrogel, the method comprising:
(i) mixing:
a hydrogel microparticle having a first crosslinking group,
a polymeric fiber having a second crosslinking group,
an initiator, and
a crosslinker,
to form a precursor composition; and
(ii) applying focused ultrasound (FUS) to the precursor composition, whereby each of the first crosslinking group and the second crosslinking group reacts with the crosslinker, thereby the hydrogel microparticle and the polymeric fiber are crosslinked to form the granular hydrogel.

2. The method of claim 1, wherein the precursor composition further comprises:
a removable particle, which upon removal changes the porosity of the granular hydrogel, and/or
a viscosity promotor, which increases viscosity upon FUS-induced heating.

3. The method of claim 2, further comprising removing the removable particle, thereby changing the porosity of the granular hydrogel.

4. The method of claim 2, where in the removable particle comprises gelatin, extracellular matrix-derived hydrogel particles, polymethylmethacrylate particles, poly alpha esters, poly ester amides, particles formed from hydrogels with enzymatically cleavable crosslinks, particles from hydrogels with physical crosslinks, alginate particles, agarose particles, pluronic, poly-NIPAAM-based particles, or combination thereof.

5. The method of claim 2, wherein the viscosity promotor comprises poly(di(ethylene glycol) methyl ether methacrylate (PDEGMA).

6. (canceled)

7. The method of claim 1, wherein each of the first each of the first crosslinking group and the second crosslinking group is independently norbornene, methacrylate, acrylate, vinyl sulfone, azide, cyclooctyne, hydrazide, aldehyde, thrombin, fibrin, or combination thereof.

8. The method of claim 1, wherein the crosslinker comprises thiol groups.

9. (canceled)

10. The method of claim 1, wherein the hydrogel microparticle comprises a hyaluronic acid; a poly(ethylene glycol) (PEG); a polynorbornene; heparin; a polysialic acid; a poly(glycerol); a poly(oxazoline); a poly(vinylpyrrolidone); a poly(acrylamide); a poly(N,Ndimethylacrylamide); a poly(acrylamide); a poly(lactic acid) (PLA); a polyglycolide (PGA); a copolymer of PLA and PGA (PLGA); a poly(vinyl alcohol) (PVA); poly(ethylene oxide); a poly(ethylene oxide)-co-poly(propylene oxide) block copolymer; a poloxamine; a polyanhydride; a polyorthoester; a poly(hydroxy acids); a polydioxanone; a polycarbonate; a polyaminocarbonate; a poly(vinyl pyrrolidone); a poly(ethyl oxazoline); a polyurethane; a carboxymethyl cellulose; a hydroxyalkylated cellulose; a polypeptide; a polypeptoid; a polysaccharide; a carbohydrate; collagen; a extracellular matrix-derived hydrogel; gelatin; alginate; dextran; a self-assembled peptide or peptide amphiphile, or combinations thereof.

11. (canceled)

12. The method of claim 1, wherein the polymeric fiber comprises a hyaluronic acid; a poly(ethylene glycol) (PEG); a polynorbornene; heparin; a polysialic acid; a poly(glycerol); a poly(oxazoline); a poly(vinylpyrrolidone); a poly(acrylamide); a poly(N,Ndimethylacrylamide); a poly(acrylamide); a poly(lactic acid) (PLA); a polyglycolide (PGA); a copolymer of PLA and PGA (PLGA); a poly(vinyl alcohol) (PVA); poly(ethylene oxide); a poly(ethylene oxide)-co-poly(propylene oxide) block copolymer; a poloxamine; a polyanhydride; a polyorthoester; a poly(hydroxy acids); a polydioxanone; a polycarbonate; a polyaminocarbonate; a poly(vinyl pyrrolidone); a poly(ethyl oxazoline); a polyurethane; a carboxymethyl cellulose; a hydroxyalkylated cellulose; a polypeptide; a polypeptoid; a polysaccharide; a carbohydrate; collagen; a extracellular matrix-derived hydrogel; gelatin; alginate; dextran; a self-assembled peptide or peptide amphiphile, or combinations thereof.

13-14. (canceled)

15. The method of claim 1, further comprising
(i-a) injecting the precursor composition from (i) into a subject; and
(ii) applying focused ultrasound (FUS) to the injected precursor composition in the subject, thereby forming a crosslinked granular hydrogel in the subject.

16. The method of claim 15, wherein at least one of the hydrogel microparticle and the polymeric fiber in the precursor composition comprises an embedded cell.

17. The method of claim 15, wherein at least one of the hydrogel microparticle and the polymeric fiber in the precursor composition comprises an embedded drug.

18-20. (canceled)

21. A method of regenerating a tissue in a subject, the method comprising:
preparing a crosslinked granular hydrogel from a precursor composition injected into the subject, the precursor composition having an embedded cell, according to the method of claim 16; and
allowing the cell to grow in the subject, thereby regenerating a tissue from the cell.

22. A method of delivering a drug to a subject, the method comprising:
preparing a crosslinked granular hydrogel from a precursor composition injected into the subject, the precursor composition having the drug embedded therein, according to the method of claim 17; and
releasing the drug from the crosslinked granular hydrogel, thereby delivering the drug to the subject.

23. The method of claim 22, wherein the precursor composition further comprises a removable particle, wherein the removable particle is embedded with a first portion of the drug, and wherein at least one of the hydrogel microparticle and the polymeric fiber is embedded with a second portion of the drug.

24. A crosslinked granular hydrogel produced by the method of claim 1.

25. (canceled)

26. A method of culturing cells, the method comprising:
mixing the cells with the scaffold of claim 25, thereby the cells are embedded in the scaffold; and
culturing the cells embedded in the scaffold.

27. An implant comprising a crosslinked granular hydrogel produced from an injected precursor composition according to the method of claim 15.

28-29. (canceled)

30. The method of claim 1, wherein the FUS is applied with a continuous duty cycle of 100%.

31. (canceled)

32. The method of claim 31, wherein the crosslinked granular hydrogel comprises a compressive moduli of about 5 kPa, about 10 kPa, or about 20 kPa.
